(19) United States
(12) Patent Application Publication
(10) Pub. No.: US 2007/0149597 A1
(43) Pub. Date: Jun. 28, 2007

(54) Medicinal composition as immunosuppressant

(75) Inventors: Takahide Nishi, Tokyo (JP); Takaichi Shimozato, Miura-shi (JP); Takashi Kagari, Yokohama-shi (JP); Hiromi Doi, Yokohama-shi (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: SANKYO COMPANY, LIMITED, Tokyo (JP)

(21) Appl. No.: 11/698,698

(22) Filed: Jan. 26, 2007

Related U.S. Application Data
(63) Continuation-in-part of application No. PCT/JP05/13843, filed on Jul. 28, 2005.

(30) Foreign Application Priority Data
Jul. 29, 2004 (JP) 2004-222420

(51) Int. Cl. A61K 31/401; A61K 31/40; A61K 31/366; A61K 31/22

(52) U.S. Cl. 514/408; 514/423; 514/460; 514/548

(57) ABSTRACT

A pharmaceutical composition useful as a preventive or therapeutic agent for immune-related diseases. The pharmaceutical composition includes at least HMG-COA reductase inhibitor and at least one amino alcohol compound having the following formula (I), a pharmacologically acceptable salt thereof, or a pharmacologically acceptable ester thereof 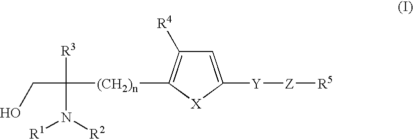
wherein R¹ and R² each represents hydrogen; R³ represents lower alkyl or hydroxymethyl; R⁴ represents hydrogen, alkyl, alkoxy or halogen; R⁵ represents hydrogen, halogeno, cyclohexyl or phenyl; X represents vinylene (CH═CH), oxygen, sulfur or methylated nitrogen; Y represents a single bond, oxygen, sulfur or carbonyl; Z represents a single bond or C₁-C₈ alkylene; n is 2 or 3.

### CROSS-REFERENCE TO RELATED APPLICATION

This application is a continuation-in-part application of International application PCT/JP2005/013843 filed Jul. 28, 2005, the entire contents of which are incorporated by reference herein.

### BACKGROUND OF THE INVENTION

1. Technical Field

The present invention relates to an excellent medicinal composition as a prophylactic or therapeutic agent for immune-related diseases such as reject reactions in organ or skin transplantation, comprising a 3-hydroxy-3-methylglutaryl coenzyme A reductase (hereinafter called as HMG-COA reductase) inhibitor and one or more than one compound selected from amino alcohol derivatives having immunosuppressive activity, pharmacologically acceptable salts thereof, and pharmacological esters thereof as active ingredients.

2. Background Information

In the past, anti-inflammatory agents such as steroids have been used for inflammatory reactions arising from abnormal immune response in the treatment of immune-related diseases such as rheumatoid arthritis and other autoimmune diseases. These are, however, a symptomatic therapy, but not a fundamental remedy. In addition, it has been reported that immune system abnormalities are also involved in the development of diabetes and nephritis (for example, see Non-patent literature 1 and Non-patent literature 2). Nevertheless, such medicaments to solve these abnormalities have never been developed.

Cyclosporin A (CsA), tacrolimus (TRL), and the like, which are previously existing immunosuppressants, are very important for preventing reject reactions in organ or skin transplantation, and for treatment or prophylaxis of various autoimmune diseases. However, these immunosuppressants are known to exert toxicities in the kidney and liver. To mitigate these side effects, concomitant treatment with immunosuppressants plus steroids is widely used. Nevertheless, significant immunosuppressive effects without adverse events are not always obtained.

In addition, although research and development of amino alcohol derivatives as immunosuppressants have recently been reported, these agents have not been widely used so far in clinical practice.

As the amino alcohol derivatives, for example, the following compounds are known:
(1) Compounds having the general formula (a) (for example, see Patent literature 1) 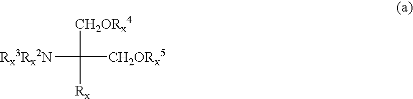
   {in the above compounds (a),

Rₓ represents a straight or branched carbon chain which may optionally be substituted with substituent (s) [said chain may contain a double bond, a triple bond, an oxygen atom, a sulfur atom, a group of formula —N(Rₓ⁶)—(wherein Rₓ⁶ represents a hydrogen atom), an arylene group which may optionally be substituted with substituent(s), or a heteroarylene group which may optionally be substituted with substituent (s), and may contain, at the end of said chain, an aryl group which may optionally be substituted with substituent(s), a cycloalkyl group which may optionally be substituted with substituent(s), or a heteroaryl group which may optionally be substituted with substituent(s)], and R ², Rx³, Rx⁴., and Rx⁵ are the same or different and each represents a hydrogen atom or an alkyl group}.
(2) Compounds having the general formula (b) (for example, see Patent literature 2) 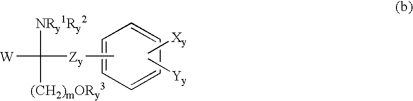
   [in the above compounds (b),

R_{y}¹, R_{y}², and R_{y}³each represent a hydrogen atom or the like; W represents a hydrogen atom, an alkyl group or the like; Z_{y} represents a single bond or an alkylene group; X_{y} represents a hydrogen atom or an alkoxy group, and Y_{y} represents a hydrogen atom, an alkyl group, an alkoxy group, an acyl group, an acyloxy group, an amino group, an acylamino group or the like].
(1) Compounds having the general formula (c) (for example, see Patent literature 3) 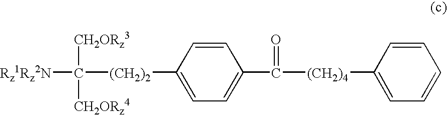
   [in the above compounds (c),

R_{z}¹, R_{z}², R_{z}³, and R_{z}⁴ are the same or different and each represents a hydrogen atom or an acyl group].
(2) Compounds having the general formula (d) (for example, see Patent literatures 4 and 5) 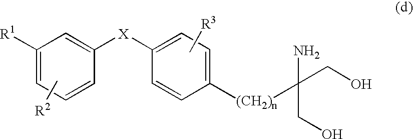
   [in the above compounds (d),

R¹ represents a halogen atom, a trihalomethyl group, a hydroxyl group, a lower alkyl group having from 1 to 7 carbon atoms, a phenoxymethyl group, or the like; R² represents a hydrogen atom, a halogen atom, a trihalomethyl group or the like; R³ represents a hydrogen atom, a halogen atom, a trihalomethyl group or the like; X represents an oxygen atom, a sulfur atom, a SO group or a S0₂ group; and n represents an integer of from 1 to 4].

On the other hand, this applicant disclosed the compounds having the following general formula (e) according to Japanese Patent Official Gazette 2002-167382 (Patent literature 6). 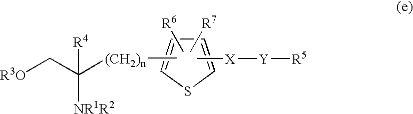
[wherein, R¹ and R² represent a hydrogen atom, or a protective group for the amino group; R³ represents a hydrogen atom, or a protective group for the hydroxyl group; R4 represents a lower alkyl group; n represents an integer of from 1 to 6; X represents an ethylene group or the like; Y represents a C₁-C₁₀ alkylene group or the like; R⁵ represents an aryl group, an aryl group substituted with substituent(s), or the like; R⁶ and R⁷ represent a hydrogen atom or the like; when R⁵ represents a hydrogen atom, however, Y represents a group other than a single bond and a straight chained C₁-C₁₀ alkylene group].

Additionally, this applicant disclosed the compounds having the following general formula (f) according to Japanese Patent Official Gazette 2003-267950 (Patent literature 7). 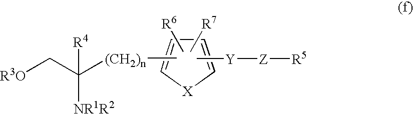
[wherein, R¹ and R2 are the same or different and each represents a hydrogen atom, a protective group for the amino group or the like; R³ represents a hydrogen atom, a protective group for the hydroxyl group or the like; R⁴ represents a lower alkyl group; n represents an integer of from 1 to 6; X represents an oxygen atom or a nitrogen atom which is not substituted or is substituted with a lower alkyl group; Y represents an ethylene group or the like; Z represents an alkylene group having from 1 to 10 carbon atoms or the like; R⁵ represents an aryl group, an aryl group which is substituted with substituent(s), or the like; R⁵ and R⁷ represent a hydrogen atom or the like; when R⁵ represents a hydrogen atom, however, Z represents a group other than a single bond and a straight chained alkylene group having from 1 to 10 carbon atoms].

Additionally, it has become widely known that the amino alcohol derivatives described above exert activities by phosphorylation of the hydroxyl group in the molecule of said derivatives in vivo.

On the other hand, an HMG-COA reductase inhibitor was recently reported to exhibit therapeutic effects against rheumatic arthritis (see Non-patent literature 3 and Non-patent literature 4). However, since the efficacy was reported in studies carried out in limited cases, it is still unclear whether HMG-COA reductase inhibitors are generally effective.

In light of this background, it is desired to discover medicinal compositions exerting excellent immunosuppressive activity with low toxicity.
[Non-patent literature 1] Kidney International, Vol. 51, 94 (1997)
[Non-patent literature 2· Journal of Immunology, vol.157,4691 (1996)
(Non-patent literature 3] Journal of Rheumatology, 2002 Sep; 29(9): 2024-6.
[Non-patent literature 4] Lupus. 2003; 12(8): 607-11.
[Patent literature 1] WO94/08943 (EP627406)
[Patent literature 2] WO96/06068
[Patent literature 3] WO98/45249
[Patent literature 4] WO03/029184
[Patent literature 5] WO03/029205
[Patent literature 6] Japanese Patent Publication (Kokai) Number 2002-167382
[Patent literature 7] Japanese Patent Publication (Kokai) Number 2003-267950

### SUMMARY OF THE INVENTION

The object of the present invention is to provide excellent medicinal compositions as a prophylactic or a therapeutic agent for immune-related diseases such as reject reactions caused by organ or skin transplantation, and autoimmune diseases such as rheumatic arthritis, psoriasis, inflammatory bowel disease, multiple sclerosis, and other autoimmune diseases.

The present inventors have eagerly studied to develop a medicinal composition having excellent immunosuppressive activity and found a medicinal composition of the present invention having excellent immunosuppressive effects and low toxicity, and that the medicinal composition augmented a pharmacological activity of each immunosuppressant contained in the medicinal composition described above, and lowered side effects of each immunosuppressant and that the medicinal composition is useful as a prophylactic or therapeutic agent for autoimmune diseases such as systemic lupus erythematosus, rheumatoid arthritis, polymyositis, dermatomyositis, scleroderma, Behcet's syndrome, Crohn's disease, ulcerative colitis, autoimmune hepatitis, a plastic anemia, idiopathic thrombocytopenic purpura, autoimmunehemolytic anemia, multiple sclerosis, autoimmunebullosis, psoriasis vulgaris, vasculitis syndrome, Wegener's granuloma, uveitis, idiopathic interstitial pneumonia, Goodpasture's syndrome, sarcoidosis, allergic granulomatous angitis, bronchial asthma, myocarditis, cardiomyopathy, aortitis syndrome, postmyocardial infarction syndrome, primary pulmonary hypertension, minimal change nephrotic syndrome, membranous nephropathy, membranoproliferative glomerulonephritis, focal glomerular sclerosis, crescentic glomerulonephritis, myasthenia gravis, inflammatory neuropathy, atopic dermatitis, chronic actinic dermatitis, acute polyarthritis, Sydenham's chorea, systemic sclerosis, adult-onset type diabetes mellitus, insulin dependent diabetes mellitus, juvenile diabetes mellitus, atherosclerosis, glomerularnephritis, tubulointerstitial nephritis, primary biliary cirrhosis, primary sclerosing cholangitis, fulminant hepatitis, viral hepatitis, GVHD, reject reactions caused by transplantation of various organs, contact dermatitis, and sepsis, or other immune-related diseases, and completed the present invention.

The present invention is directed to the following:
(1) a medicinal composition of the present invention is a medicinal composition comprising at least one agent selected from the group consisting of HMG-COA reductase inhibitors and at least one compound selected from the group consisting of amino alcohol derivatives having the general formula (I), pharmacologically acceptable salts thereof, and pharmacological esters thereof: 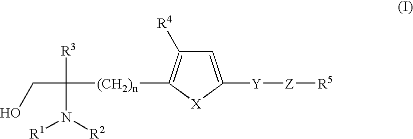
   [wherein, R¹ and R² are the same or different and each represents a hydrogen atom, or a lower alkyl group,
R³ represents a lower alkyl group or a hydroxymethyl group,
R⁴ represents a hydrogen atom, a lower alkyl group, a lower alkoxy group or a halogen atom,
R⁵ represents a hydrogen atom, a halogen atom, a cyclohexyl group, a phenyl group, or a phenyl group substituted with from 1 to 3 substituents selected from Substituent group a,
X represents a vinylene group (CH=CH group) , an oxygen atom, a sulfur atom, or a methylated nitrogen atom,
Y represents a single bond, an oxygen atom, a sulfur atom or a carbonyl group,
Z represents a single bond, an alkylene group having from 1 to 8 carbon atoms, or an alkylene group having from 1 to 8 carbon atoms which is substituted with from 2 to 8 fluorine atoms,
n represents an integer of 2 or 3, and
Substituent group a represents the group consisting of a halogen atom, a cyano group, a lower alkyl group, a lower cycloalkyl group, a lower alkoxy group, a trifluoromethyl group, a phenyl group, and a benzyloxy group.

Of the medicinal compositions according to (1) described above, preferred medicinal compositions are as follows:
(2) a medicinal composition according to (1) described above, wherein an amino alcohol derivative having the general formula (I) is a compound having the general formula (Ia) shown below, 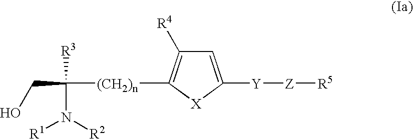
(3) a medicinal composition according to (1) or (2) described above, wherein the HMG-COA reductase inhibitor is at least one agent selected from the group consisting of pravastatin, lovastatin, simvastatin, fluvastatin, atorvastatin, rosuvastatin and pitavastatin,
(4) a medicinal composition according to (1) or (2) described above, wherein the HMG-CoA reductase inhibitor is at least one agent selected from the group consisting of pravastatin, simvastatin and atorvastatin,
(5) a medicinal composition according to (1) or (2) described above, wherein the HMG-COA reductase inhibitor is pravastatin,
(6) a medicinal composition according to (1) or (2) described above, wherein the HMG-COA reductase inhibitor is atorvastatin,
(7) a medicinal composition according to any one selected from (1) to (6) described above, which comprises an amino alcohol derivative having the general formula (I) wherein R¹ and R² are a hydrogen atom,
(8) a medicinal composition according to any one selected from (1) to (7) described above, which comprises an amino alcohol derivative having the general formula (I) wherein R³ is a methyl, ethyl, or hydroxymethyl group,
(9) a medicinal composition according to any one selected from (1) to (7) described above, which comprises an amino alcohol derivative having the general formula (I) wherein R3is a methyl group or an ethyl group,
(10) a medicinal composition according to any one selected from (1) to (7) described above, which comprises an amino alcohol derivative having the general formula (I) wherein R³ is a hydroxymethyl group,
(11) a medicinal composition according to any one selected from (1) to (7) described above, which comprises an amino alcohol derivative having the general formula (I) wherein R³ is a methyl group,
(12) a medicinal composition according to any one selected from (1) to (11) described above, which comprises an amino alcohol derivative having the general formula (I) wherein R⁴ is a hydrogen atom,
(13) a medicinal composition according to any one selected from (1) to (11) described above, which comprises an amino alcohol derivative having the general formula (I) wherein R⁴ is a chlorine atom,
(14) a medicinal composition according to any one selected from (1) to (13) described above, which comprises an amino alcohol derivative having the general formula (I) wherein R is a phenyl group or a phenyl group substituted with from 1 to 3 substituents selected from Substituent group a,
(15) a medicinal composition according to any one selected from (1) to (13) described above, which comprises an amino alcohol derivative having the general formula (I) wherein R is a phenyl group substituted with from 1 to 3 substituents selected from the group consisting of a fluorine atom, a cyano group, a methyl group, a methoxy group, an ethoxy group and a phenyl group,
(16) a medicinal composition according to any one selected from (1) to (13) described above, which comprises an amino alcohol derivative having the general formula (I) wherein R is a phenyl group substituted with a substituent selected from the group consisting of a fluorine atom, a cyano group, a methyl group, a methoxy group, an ethoxy group and a phenyl group,
(17) a medicinal composition according to any one selected from (1) to (13) described above, which comprises an amino alcohol derivative having the general formula (I) wherein R is a phenyl group of which the para position is substituted with a substituent selected from the group consisting of a fluorine atom, a cyano group, a methyl group, a methoxy group, an ethoxy group and a phenyl group,
(18) a medicinal composition according to any one selected from (1) to (13) described above, which comprises an amino alcohol derivative having the general formula (I) wherein R⁵ is a phenyl group substituted with a trifluoromethyl group or a benzyloxy group,
(19) a medicinal composition according to any one selected from (1) to (13) described above, which comprises an amino alcohol derivative having the general formula (I) wherein R⁵is a phenyl group of which the meta position is substituted with a trifluoromethyl group or a benzyloxy group,
(20) a medicinal composition according to any one selected from (1) to (13) described above, which comprises an amino alcohol derivative having the general formula (I) wherein R⁵is a phenyl group substituted with from 1 to 3 substituents selected from the group consisting of a lower alkyl group and a lower alkoxy group,
(21) a medicinal composition according to any one selected from (1) to (13) described above, which comprises an amino alcohol derivative having the general formula (I) wherein R⁵is a phenyl group substituted with from 1 to 3 substituents selected from the group consisting of a methyl group and a methoxy group,
(22) a medicinal composition according to any one selected from (1) to (13) described above, which comprises an amino alcohol derivative having the general formula (I) wherein R⁵ is a phenyl group,
(23) a medicinal composition according to any one selected from (1) to (13) described above, which comprises an amino alcohol derivative having the general formula (I) wherein R⁵ is a hydrogen atom,
(24) a medicinal composition according to any one selected from (1) to (23) described above, which comprises an amino alcohol derivative having the general formula (I) wherein X is a vinylene group (CH═CH group),
(25) a medicinal composition according to any one selected from (1) to (23) described above, which comprises an amino alcohol derivative having the general formula (I) wherein X is an oxygen atom, a sulfur atom or a nitrogen atom substituted with a methyl group,
(26) a medicinal composition according to any one selected from (1) to (23) described above, which comprises an amino alcohol derivative having the general formula (I) wherein X is a nitrogen atom substituted with a methyl group,
(27) a medicinal composition according to any one selected from (1) to (26) described above, which comprises an amino alcohol derivative having the general formula (I) wherein Y represents a single bond,
(28) a medicinal composition according to any one selected from (1) to (26) described above, which comprises an amino alcohol derivative having the general formula (I) wherein Y represents an oxygen atom,
(29) a medicinal composition according to any one selected from (1) to (26) described above, which comprises an amino alcohol derivative having the general formula (I) wherein Y represents a sulfur atom,
(30) a medicinal composition according to any one selected from (1) to (26) described above, which comprises an amino alcohol derivative having the general formula (I) wherein Y represents a carbonyl group,
(31) a medicinal composition according to any one selected from (1) to (30) described above, which comprises an amino alcohol derivative having the general formula (I) wherein Z represents a single bond or an alkylene group having from 1 to 8 carbon atoms,
(32) a medicinal composition according to any one selected from (1) to (30) described above, which comprises an amino alcohol derivative having the general formula (I) wherein Z represents a single bond,
(33) a medicinal composition according to any one selected from (1) to (30) described above, which comprises an amino alcohol derivative having the general formula (I) wherein Z represents an alkylene group having from 1 to 8 carbon atoms,
(34) a medicinal composition according to any one selected from (1) to (30) described above, which comprises an amino alcohol derivative having the general formula (I) wherein Z represents an alkylene group having 8 carbon atoms,
(35) a medicinal composition according to any one selected from (1) to (30) described above, which comprises an amino alcohol derivative having the general formula (I) wherein Z represents an alkylene group having from 2 to 5 carbon atoms,
(36) a medicinal composition according to any one selected from (1) to (30) described above, which comprises an amino alcohol derivative having the general formula (I) wherein Z represents an alkylene group having 3 carbon atoms,
(37) a medicinal composition according to any one selected from (1) to (36) described above, which comprises an amino alcohol derivative having the general formula (I) wherein n represents an integer of 2,
(38) a medicinal composition according to any one selected from (1) to (36) described above, which comprises an amino alcohol derivative having the general formula (I) wherein n represents an integer of 3,
(39) a medicinal composition according to any one selected from (1) to (6) described above, wherein an amino alcohol derivative having the general formula (I) is the following compound:
2-amino-2-methyl-4-{1-methyl-5-[4-(2-methylphenyl)butanoyl]pyrrol-2-yl}butan-1-ol,
2-amino-2-methyl-4-{1-methyl-5-[4-(3-methylphenyl)butanoyl]pyrrol-2-yl}butan-1-ol,
2-amino-2-methyl-4-{1-methyl-5-[4-(4-methylphenyl)butanoyl]pyrrol-2-yl}butan-1-ol,
2-amino-2-methyl-4-{1-methyl-5-[4-(2,3-dimethylphenyl)butanoyl]pyrrol-2-yl}butan-1-ol,
2-amino-2-methyl-4-{1-methyl-5-[4-(2,4-dimethylphenyl)butanoyl]pyrrol-2-yl}butan-1-ol,
2-amino-2-methyl-4-{1-methyl-5-[4-(2,5-dimethylphenyl)butanoyl]pyrrol-2-yl}butan-1-ol,
2-amino-2-methyl-4-{1-methyl-5-[4-(3,4-dimethylphenyl)butanoyl]pyrrol-2-yl}butan-1-ol,
2-amino-2-methyl-4-{1-methyl-5-[4-(3,5-dimethylphenyl)butanoyl]pyrrol-2-yl}butan-1-ol,
2-amino-2-methyl-4-{1-methyl-5-[4-(4-ethylphenyl)butanoyl]pyrrol-2-yl}butan-1-ol,
2-amino-2-methyl-4-{1-methyl-5-[4-(4-isopropylphenyl)butanoyl]pyrrol-2-yl}butan-1-ol,
2-amino-2-methyl-4-{1-methyl-5-[4-(4-cyclopropylphenyl)butanoyl]pyrrol-2-yl}butan-1-ol,
2-amino-2-methyl-4-{1-methyl-5-[4-(4-t-butylphenyl)butanoyl]pyrrol-2-yl}butan-1-ol,
2-amino-2-methyl-4-{1-methyl-5-[4-(4-methoxyphenyl)butanoyl]pyrrol-2-yl}butan-1-ol,
2-amino-2-methyl-4-{1-methyl-5-[4-(3,4-dimethoxyphenyl)butanoyl]pyrrol-2-yl}butan-1-ol,
2-amino-2-methyl-4-{1-methyl-5-[4-(3-methyl-4-methoxyphenyl) butanoyl]pyrrol-2-yl}butan-1-ol,
2-amino-2-methyl-4-{1-methyl-5-[4-(3-methoxy-4-methylphenyl) butanoyl]pyrrol-2-yl}butan-1-ol,
2-amino-2-methyl-4-{1-methyl-5-[4-(4-fluorophenyl)butanoyl]pyrrol-2-yl}butan-1-ol,
2-amino-2-methyl-4-{1-methyl-5-[4-(4-chlorophenyl)butanoyl]pyrrol-2-yl}butan-1-ol,
2-amino-2-methyl-4-{1-methyl-5-[4-(4-trifluoromethylphenyl) butanoyl]pyrrol-2-yl}butan-1-ol,
2-amino-2-methyl-4-{1-methyl-5-[4-(4-cyanophenyl)butanoyl]pyrrol-2-yl}butan-1-ol,
2-amino-2-ethyl-4-{1-methyl-5-[4-(2-methylphenyl)butanoyl]pyrrol-2-yl}butan-1-ol,
2-amino-2-ethyl-4-{1-methyl-5-[4-(3-methylphenyl)butanoyl]pyrrol-2-yl}butan-1-ol,
2-amino-2-ethyl-4-{1-methyl-5-[4-(4-methylphenyl)butanoyl]pyrrol-2-yl}butan-1-ol,
2-amino-2-ethyl-4-{1-methyl-5-[4-(2,3-dimethylphenyl)butanoyl]pyrrol-2-yl}butan-1-ol,
2-amino-2-ethyl-4-{1-methyl-5-[4-(2,4-dimethylphenyl)butanoyl]pyrrol-2-yl}butan-1-ol,
2-amino-2-ethyl-4-{1-methyl-5-[4-(2,5-dimethylphenyl)butanoyl]pyrrol-2-yl}butan-1-ol,
2-amino-2-ethyl-4-{1-methyl-5-[4-(3,4-dimethylphenyl)butanoyl]pyrrol-2-yl}butan-1-ol,
2-amino-2-ethyl-4-{1-methyl-5-[4-(3,5-dimethylphenyl)butanoyl]pyrrol-2-yl}butan-1-ol,
2-amino-2-ethyl-4-{1-methyl-5-[4-(4-ethylphenyl)butanoyl]pyrrol-2-yl}butan-1-ol,
2-amino-2-ethyl-4-{1-methyl-5-[4-(4-isopropylphenyl)butanoyl]pyrrol-2-yl}butan-1-ol,
2-amino-2-ethyl-4-{1-methyl-5-(4-(4-cyclopropylphenyl)butanoyl]pyrrol-2-yl}butan-1-ol,
2-amino-2-ethyl-4-{1-methyl-5-[4-(4-t-butylphenyl)butanoyl]pyrrol-2-yl}butan-1-ol,
2-amino-2-ethyl-4-{1-methyl-5-[4-(4-methoxyphenyl)butanoyl]pyrrol-2-yl}butan-1-ol,
2-amino-2-ethyl-4-{1-methyl-5-[4-(3,4-dimethoxyphenyl)butanoyl]pyrrol-2-yl}butan-1-ol,
2-amino-2-ethyl-4-{1-methyl-5-[4-(3-methyl-4-methoxyphenyl) butanoyl]pyrrol-2-yl}butan-1-ol,
2-amino-2-ethyl-4-{1-methyl-5-[4-(4-fluorophenyl)butanoyl]pyrrol-2-yl}butan-1-ol,
2-amino-2-ethyl-4-{1-methyl-5-[4-(4-chlorophenyl)butanoyl]pyrrol-2-yl}butan-1-ol,
2-amino-2-ethyl-4-{1-methyl-5-[4-(4-trifluoromethylphenyl) butanoyl]pyrrol-2-yl}butan-1-ol,
2-amino-2-ethyl-4-{1-methyl-5-[4-(4-cyanophenyl)butanoyl]pyrrol-2-yl}butan-1-ol,
2-amino-2-methyl-4-{1-methyl-5-[5-(2-methylphenyl)pentanoyl]pyrrol-2-yl}butan-1-ol,
2-amino-2-methyl-4-{1-methyl-5-[5-(3-methylphenyl)pentanoyl]pyrrol-2-yl}butan-1-ol,
2-amino-2-methyl-4-{1-methyl-5-[5-(4-methylphenyl)pentanoyl]pyrrol-2-yl}butan-1-ol,
2-amino-2-methyl-4-{1-methyl-5-[5-(2,3-dimethylphenyl)pentanoyl]pyrrol-2-yl}butan-1-ol,
2-amino-2-methyl-4-{1-methyl-5-[5-(2,4-dimethylphenyl)pentanoyl]pyrrol-2-yl}butan-1-ol,
2-amino-2-methyl-4-{1-methyl-5-[5-(2,5-dimethylphenyl)pentanoyl]pyrrol-2-yl}butan-1-ol,
2-amino-2-methyl-4-{1-methyl-5-[5-(3,4-dimethylphenyl)pentanoyl]pyrrol-2-yl}butan-1-ol,
2-amino-2-methyl-4-{1-methyl-5-[5-(3,5-dimethylphenyl)pentanoyl]pyrrol-2-yl}butan-1-ol,
2-amino-2-methyl-4-{1-methyl-5-[5-(3-methyl-4-methoxyphenyl) pentanoyl]pyrrol-2-yl}butan-1-ol,
2-mino-2-methyl-4-{1-methyl-5-[5-(3-methoxy-4-methylphenyl) pentanoyl]pyrrol-2-yl}butan-1-ol, or
2-amino-2-methyl-4-{1-methyl-5-[5-(4-cyanophenyl)pentanoyl]pyrrol-2-yl}butan-1-ol,
(40) a medicinal composition according to any one selected from (1) to (6) described above, wherein an amino alcohol derivative having the general formula (I) is the following compound:
2-amino-2-methyl-4-{1-methyl-5-[4-(2-methylphenyl)butanoyl]pyrrol-2-yl}butan-1-ol,
2-amino-2-methyl-4-{1-methyl-5-[4-(3-methylphenyl)butanoyl]pyrrol-2-yl}butan-1-ol,
2-amino-2-methyl-4-{1-methyl-5-[4-(4-methylphenyl)butanoyl]pyrrol-2-yl}butan-1-ol,
2-amino-2-methyl-4-{1-methyl-5-[4-(2,3-dimethylphenyl)butanoyl]pyrrol-2-yl}butan-1-ol,
2-amino-2-methyl-4-{1-methyl-5-[4-(2,4-dimethylphenyl)butanoyl]pyrrol-2-yl}butan-1-ol,
2-amino-2-methyl-4-{1-methyl-5-[4-(2,5-dimethylphenyl)butanoyl]pyrrol-2-yl}butan-1-ol,
2-amino-2-methyl-4-{1-methyl-5-[4-(3,4-dimethylphenyl)butanoyl]pyrrol-2-yl}butan-1-ol,
2-amino-2-methyl-4-{1-methyl-5-[4-(3,5-dimethylphenyl)butanoyl]pyrrol-2-yl}butan-1-ol,
2-amino-2-methyl-4-{1-methyl-5-[4-(3-methyl-4-methoxyphenyl) butanoyl]pyrrol-2-yl}butan-1-ol,
2-amino-2-methyl-4-{1-methyl-5-[4-(3-methoxy-4-methylphenyl) butanoyl]pyrrol-2-yl}butan-1-ol,
2-amino-2-methyl-4-{1-methyl-5-[4-(4-cyanophenyl)butanoyl]pyrrol-2-yl}butan-1-ol,
2-amino-2-methyl-4-{1-methyl-5-[5-(2-methylphenyl)pentanoyl]pyrrol-2-yl}butan-1-ol,
2-amino-2-methyl-4-{1-methyl-5-[5-(3-methylphenyl)pentanoyl]pyrrol-2-yl}butan-1-ol,
2-amino-2-methyl-4-{1-methyl-5-[5-(4-methylphenyl)pentanoyl]pyrrol-2-yl}butan-1-ol,
2-amino-2-methyl-4-{1-methyl-5-[5-(2,3-dimethylphenyl)pentanoyl]pyrrol-2-yl}butan-1-ol,
2-amino-2-methyl-4-{1-methyl-5-[5-(2,4-dimethylphenyl)pentanoyl]pyrrol-2-yl}butan-1-ol,
2-amino-2-methyl-4-{1-methyl-5-[5-(2,5-dimethylphenyl)pentanoyl]pyrrol-2-yl}butan-1-ol,
2-amino-2-methyl-4-{1-methyl-5-[5-(3,4-dimethylphenyl)pentanoyl]pyrrol-2-yl}butan-1-ol,
2-amino-2-methyl-4-{1-methyl-5-[5-(3,5-dimethylphenyl)pentanoyl]pyrrol-2-yl}butan-1-ol,
2-amino-2-methyl-4-{1-methyl-5-[5-(3-methyl-4-methoxyphenyl) pentanoyl]pyrrol-2-yl}butan-1-ol,
2-amino-2-methyl-4-{1-methyl-5-[5-(3-methoxy-4-methylphenyl) pentanoyl]pyrrol-2-yl}butan-1-ol, or
2-amino-2-methyl-4-{1-methyl-5-[5-(4-cyanophenyl)pentanoyl]pyrrol-2-yl}butan-1-ol,
(41) a medicinal composition according to any one selected from (1) to (6) described above, wherein an amino alcohol derivative having the general formula (I) is the following compound:
2-amino-2-[2-(4-octylphenyl)ethyl]propan-1,3-diol,
2-amino-2-[2-(4-heptyloxyphenyl)ethyl]propan-1,3-diol,
2-amino-2-{2-[4-(5-phenylpentanoyl)phenyl]ethyl}propan-l,3-diol,
2-amino-2-{2-[4-(5-cyclohexylpentanoyl)phenyl]ethyl}propan-1,3-diol,
2-amino-2-{2-[4-(7-phenylheptanoyl)phenyl]ethyl}propan-1,3-diol,
2-amino-2-[2-(4-[2-(4-methoxyphenyl)ethoxy]phenyl)ethyl]propan-1,3-diol,
2-amino-2-[2-(4-[2-(4-ethoxyphenyl)ethoxy]phenyl)ethyl]propan-1,3-diol,
2-amino-2-[2-(4-[2-(3-fluoro-4-methoxyphenyl)ethoxy]phenyl) ethyl]propan-1,3-diol,
2-amino-2-methyl-4-[4-(4,4,5,5,5-pentafluoropentoxy)phenyl]butan-1-ol,
2-amino-2-methyl-4-[4-(3-biphenyl-4-ylpropoxy)phenyl]butan-1-ol,
2-amino-2-methyl-4-[4-(3-biphenyl-4-ylpropionyl)phenyl]butan-1-ol,
2-amino-2-methyl-4-[3-methoxy-4-(4-phenylbutoxy)phenyl]butan-1-ol,
2-amino-2-methyl-4-[4-(5-phenylpentoxy)phenyl]butan-1-ol,
2-amino-2-methyl-4-[4-(5-phenylpentanoyl)phenyl]butan-1-ol,
2-amino-2-methyl-4-(4-hexyloxyphenyl)butan-1-ol,
2-amino-2-methyl-4-[4-(3-phenylpropoxy)phenyl]butan-1-ol,
2-amino-2-methyl-4-[4-(3-cyclohexylpropoxy)phenyl]butan-1-ol,
2-amino-2-methyl-4-[4-(5-cyclohexylpentanoyl)phenyl]butan-1-ol,
2-amino-2-methyl-4-[4-heptyloxyphenyl]butan-1-ol,
2-amino-2-[4-(3-benzyloxyphenoxy)-2-chlorophenyl]propan-1,3-diol,
2-amino-2-[4-(3-benzyloxyphenylthio)-2-chlorophenyl]propan-1,3-diol,
2-amino-2-methyl-5-[4-(3-benzyloxyphenoxy)-2-chlorophenyl]pentan-1-ol, or
2-amino-2-methyl-5-[4-(3-benzyloxyphenylthio)-2-chlorophenyl]pentan-1-ol, and
(42) a medicinal composition according to (1) described above, wherein the HMG-COA reductase inhibitor is atorvastatin, and an amino alcohol derivative having the general formula (I) is
2-amino-2-[2-(4-octylphenyl)ethyl]propan-1,3-diol.

Additionally, the present invention provides
(43) a method for prophylaxis or treatment of an autoimmune disease, which is characterized by administration to mammals of an effective amount of a medicinal composition according to any one selected from (1) to (42) described above,
(44) use of a medicinal composition according to any one selected from (1) to (42) described above for preparing a therapeutic agent for autoimmune diseases,
(45) a preparation method of a medicinal composition according to any one selected from (1) to (42) described above, which comprises at least one agent selected from the group consisting of HMG-COA reductase inhibitors and at least one compound selected from the group consisting of amino alcohol derivatives having the general formula (I), pharmacologically acceptable salts thereof and pharmacologically acceptable esters thereof,
(46) an administration procedure by which at least one agent selected from the group consisting of HMG-COA reductase inhibitors and at least one compound selected from the group consisting of amino alcohol derivatives having the general formula (I), pharmacologically acceptable salts thereof and pharmacologically acceptable esters thereof are administered simultaneously, sequentially or separately on different occasions,
(47) a therapeutic agent for an autoimmune disease comprising a medicinal composition according to (1) to (42) described above, which is characterized by being a combination drug, and
(48) a therapeutic agent for an autoimmune disease comprising a medicinal composition according to (1) to (42) described above, which is characterized by being a kit comprising a pharmaceutical preparation of a drug containing at least one agent selected from the group consisting of HMG-COA reductase inhibitors and a pharmaceutical preparation of a drug containing at least one compound selected from the group consisting of amino alcohol derivatives having the general formula (I), pharmacologically acceptable salts thereof and pharmacologically acceptable esters thereof.

The HMG-COA reductase inhibitors that are one of the active ingredients of the medicinal composition of the present invention encompass all natural materials originated from organisms, semi-synthesized compounds derived from said natural materials and wholly synthesized compounds thereof, and are, for example,
(+)-(3R,5R)-3,5-dihydroxy-7-[(lS,2S,6S,8S,8aR)-6-hydroxy-2-methyl-8-[(S)-2-methyl-butyryloxy]-1,2,6,7,8,8a-hexahydro-1-naphthyl]heptanoic acid (Pravastatin, Japanese Patent Publication (Kokai) Sho 57-2240 (U.S. Pat. No. 4,346,227)),
(+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl (S)-2-methylbutyrate (Lovastatin, Japanese Patent Publication (Kokai) Sho 57-163374 (U.S. Pat. No. 4,231,938)),
(+)-(lS,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl 2,2-dimethylbutyrate (Simvastatin, Japanese Patent Publication (Kokai) Sho 56-122375 (U.S. Pat. No. 4,444,784)),
(+)-(3R*, 5S*, 6E)-7- [3-(4-fluorophenyl)-1-(1-methylethyl)-1H-indol-2-yl]-3, 5-dihydroxy-6-heptenoic acid (Fluvastatin, Japanese Patent Publication (Kokai) Sho 60-500015 (U.S. Pat. No. 4,739,073)), (3R,5S)-7-[2-(4-fluorophenyl)-5-(1-methylethyl)-3-phenyl-4-phenylaminocarbonyl-1H-pyrrol-1-yl]-3,5-dihydroxyheptenoic acid (Atorvastatin, Japanese Patent Publication (Kokai) Hei 3-58967 (U.S. Pat. No. 5,273,995)),
(+)-(3R,5S)-7-[4-(4-fluorophenyl)-6-isopropyl-2-(N-methyl-N-methanesulfonylamino)-pyrimidin-5-yl]-3,5-dihydroxy-6(E)-heptenoic acid (Rosuvastatin, Japanese Patent Publication (Kokai) Hei 5-178841 (U.S. Pat. No. 5,260,440)) or
(E)-3,5-dihydroxy-7-[4′-(4″-fluorophenyl)-2′-cyclopropylquinolin-3′-yl]-6-heptenoic acid (Pitavastatin, Japanese Patent Publication (Kokai) Hei 1-279866 (U.S. Pat. No. 5,854,259 and U.S. Pat. No. 5,856,336)).

### DETAILED DESCRIPTION OF THE INVENTION

The HMG-COA reductase inhibitor is preferably pravastatin, lovastatin, simvastatin, fluvastatin, atorvastatin, rosuvastatinor pitavastatin, more preferably pravastatin, simvastatin or atorvastatin, most preferably pravastatin or atorvastatin, and particularly preferably atorvastatin.

Planar chemical structures of representative HMG-COA reductase inhibitors are shown below:

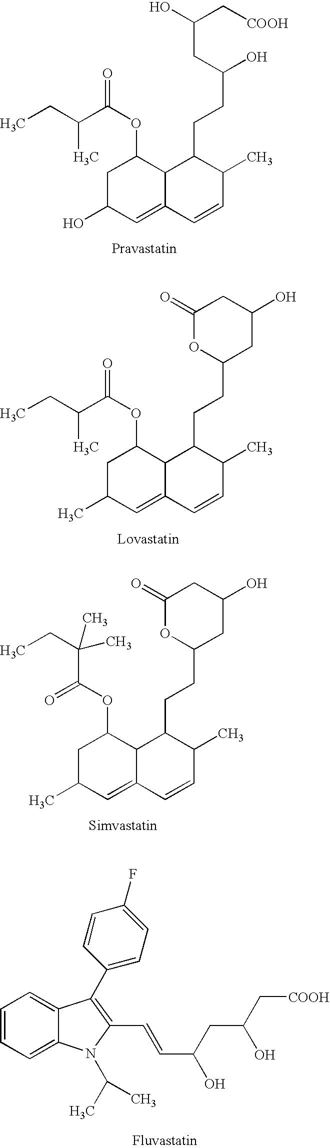

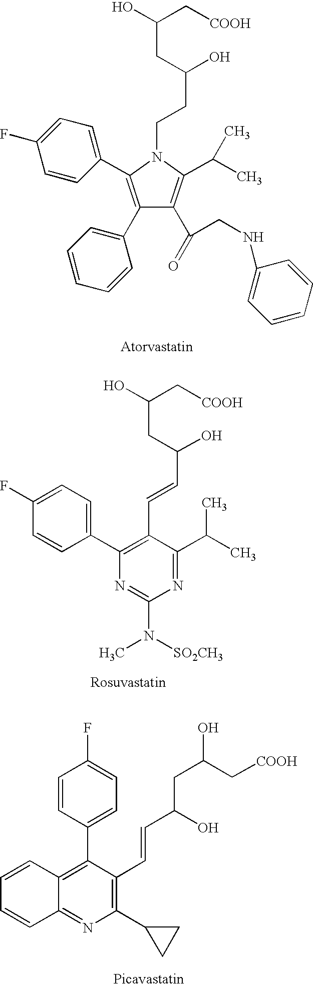

The amino alcohol derivatives, which are one of active ingredients of the medicinal composition of the present invention, have the following general formula (I).

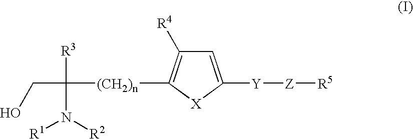

In the formula shown above, the “lower alkyl group” in the definition of R¹, R², R³, R⁴and Substituent group a can be, for example, a methyl, ethyl, propyl, isopropyl, butyl, isobutyl, s-butyl, t-butyl, pentyl or hexyl group, and is preferably a methyl, ethyl or propyl group, and more preferably a methyl or ethyl group. In the formula shown above, the “halogen atom” in the definition of R⁴, R⁵ and Substituent group a is a fluorine atom, a chlorine atom, a bromine atom or an iodine atom, and preferably a fluorine atom or a chlorine atom.

In the formula shown above, the “lower alkoxy group” in the definition of R⁴ and Substituent group a is, for example, a methoxy, ethoxy, propoxy, isopropoxy, butoxy, isobutoxy, s-butoxy, t-butoxy, pentoxy or hexyloxy group.

In the formula shown above, the “alkylene group having from 1 to 8 carbon atoms” in the definition of Z can be, for example, a methylene, ethylene, propylene, tetramethylene, pentamethylene, hexamethylene, heptamethylene, or octamethylene group, and is preferably an alkylene group having from 2 to 8 carbon atoms, and more preferably an ethylene, propylene, tetramethylene, heptamethylene, or octamethylene group.

In the formula shown above, the “alkylene group having from 1 to 8 carbon atoms which is substituted with from 2 to 8 fluorine atoms” in the definition of Z can be, for example, a difluoromethylene, 1,1-difluoroethylene, 1,1,2,2-tetrafluoroethylene, 1,1-difluoropropylene, 1,1,2,2-tetrafluoropropylene, 1,1-difluorotetramethylene, 1,1,2,2-tetrafluorotetramethylene, 1,1-difluoropentamethylene or 1,1,2,2-tetrafluoropentamethylene group, and is preferably a 1,1-difluoropropylene, 1,1,2,2-tetrafluoropropylene, 1,1-difluorotetramethylene, 1,1,2,2-tetrafluorotetramethylene, 1,l-difluoropentamethylene or 1,1,2,2-tetrafluoropentamethylene group.

In the formula shown above, the “cycloalkyl group” in the definition of Substituent group a can be, for example, a cyclopropyl, cyclobutyl, cyclopentyl or cyclohexyl group, and is preferably a cyclohexyl group.

The “pharmacologically acceptable salt thereof” described hereinbefore means a salt which can be prepared by reacting the compounds with an acid, as the compounds having the general formula (I) described hereinbefore have an amino group as a basic group. Such salt can be, for example, an inorganic acid salt, including a hydrohalide such as hydrofluoride, hydrochloride, hydrobromide, or hydroiodide, a nitrate, a perchlorate, a sulfate, a phosphate or the like; an organic acid salt, including a lower alkanesulfonate such asmethanesulfonate, trifluoromethanesulfonate, orethanesulfonate, an arylsulfonate such as benzenesulfonate or p-toluenesulfonate, an acetate, a malate, a fumarate, a succinate, a citrate, an ascorbate, a tartrate, an oxalate, a maleate, or the like; or an amino acid salt such as glycine salt, lysine salt, arginine salt, ornithine salt, glutamic acid salt, or aspartic acid salt, and is preferably hydrochloride, an acetate, a fumarate, a succinate or a maleate.

The “pharmacologically acceptable ester thereof” described hereinbefore means an ester of a compound having the general formula (I) described hereinbefore which has a hydroxyl group capable being esterified, and each ester residual group belongs to a “general protective group for the hydroxyl group”.

The “general protective group for the hydroxyl group” means a “protective group in chemical reactions” which can be cleaved by a chemical process such as hydrogenolysis, hydrolysis, electrolysis or photolysis, and a “protective group which can be removed by a biological process such as hydrolysis in vivo”.

Such the “protective group in chemical reactions” can be, for example, an “aliphatic acyl group”, including an alkylcarbonyl group such as a formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl or henicosanoyl group, an alkylcarbonyl group substituted with a carboxyl group such as a succinoyl, glutaroyl or adipoyl group, a halogeno lower alkylcarbonyl group such as a chloroacetyl, dichloroacetyl, trichloroacetyl or trifluoroacetyl group, a lower alkylcarbonyl group substituted with lower alkoxy group(s) such as a methoxyacetyl group, and an unsaturated alkylcarbonyl group such as a (E)-2-methyl-2-butenoyl group; an “aromatic acyl group”, including an arylcarbonyl group such as a benzoyl, α-naphthoyl or β-naphthoyl group, a halogeno arylcarbonyl group such as a 2-bromobenzoyl or 4-chlorobenzoyl group, an arylcarbonyl group substituted with lower alkyl groups such as a 2,4,6-trimethylbenzoyl or 4-toluoyl group, a lower alkoxylated arylcarbonyl group such as a 4-anisoyl group, an arylcarbonyl group substituted with carboxyl group(s) such as a 2-carboxybenzoyl, 3-carboxybenzoyl or 4-carboxybenzoyl group, a nitrated arylcarbonyl group such as a 4-nitrobenzoyl or 2-nitrobenzoyl group, an arylcarbonyl group substituted with lower alkoxycarbonyl group(s) such as a 2- (methoxycarbonyl) benzoyl group, and an arylcarbonyl group substituted with aryl group(s) such as a 4-phenylbenzoyl group; a “tetrahydropyranyl or tetrahydrothiopyranyl group” such as a tetrahydropyran-2-yl, 3-bromotetrahydropyran-2-yl, 4-methoxytetrahydropyran-4-yl, tetrahydrothiopyran-2-yl or 4-methoxytetrahydrothiopyran-4-yl group; a “tetrahydrofuranyl or tetrahydrothiofuranyl group” such as a tetrahydrofuran-2-yl or tetrahydrothiofuran-2-yl group; a “silyl group”, including a tri-lower alkylsilyl group such as a trimethylsilyl, triethylsilyl, isopropyldimethylsilyl, t-butyldimethylsilyl, methyldiisopropylsilyl, methyls-t-butylsilyl or triisopropylsilyl group and a tri-lower alkylsilyl group substituted with 1 or 2 aryl groups such as a diphenylmethylsilyl, diphenylbutylsilyl, diphenylisopropylsilyl or phenyldiisopropylsilyl group; an “alkoxymethyl group”, including a lower alkoxymethyl group such as a methoxymethyl, 1,1-dimethyl-1-methoxymethyl, ethoxymethyl, propoxymethyl, isopropoxymethyl, butoxymethyl or t-butoxymethyl group, a lower alkoxymethyl group substituted with lower alkoxy group(s) such as a 2-methoxyethoxymethyl group, and a halogeno lower alkoxymethyl group such as a 2,2,2-trichloroethoxymethyl or bis(2-chloroethoxy)methyl group; a “substituted ethyl group”, including a lower alkoxylated ethyl group such as a 1-ethoxyethyl or 1- (isopropoxy) ethyl group, and a halogenated ethyl group such as a 2,2,2-trichloroethyl group; an “aralkyl group”, including a lower alkyl group substituted with from 1 to 3 aryl groups such as a benzyl, α-naphthylmethyl, β-naphthylmethyl, diphenylmethyl, triphenylmethyl, a-naphthyldiphenylmethyl or 9-anthrylmethyl group, and a lower alkyl group substituted with from 1 to 3 aryl groups, of which an aryl ring is substituted with lower alkyl group(s), lower alkoxy group(s), halogen atom(s) or cyano group(s), such as a 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl, 4-cyanobenzyl, methyl, or piperonyl group; an “alkoxycarbonyl group”, including a lower alkoxycarbonyl group such as a methoxycarbonyl, ethoxycarbonyl, t-butoxycarbonyl or isobutoxycarbonyl group, and a lower alkoxycarbonyl group substituted with halogen atom(s) or tri-lower alkylsilyl group(s) such as a 2,2,2-trichloroethoxycarbonyl or 2-trimethylsilylethoxycarbonylgroup; an “alkenyloxycarbonylgroup” such as a vinyloxycarbonyl or allyloxycarbonyl group; an “aralkyloxycarbonyl group”, of which an aryl ring may be substituted with 1 or 2 substituents selected from a lower alkoxy group or a nitro group, such as a benzyloxycarbonyl, 4-methoxybenzyloxycarbonyl, 3,4-dimethoxybenzyloxycarbonyl, 2-nitrobenzyloxycarbonyl, or 4-nitrobenzyloxycarbonyl group, and is preferably an aliphatic acyl group.

On the other hand, the “protective group which can be removed by a biological process such as hydrolysis in vivo” can be, for example, an acyloxyalkyl group such as an ethylcarbonyloxymethyl, pivaloyloxymethyl, dimethylaminoacetyloxymethyl, or l-acetoxyethyl group; a 1-(alkoxycarbonyloxy)alkyl group such as a 1-(methoxycarbonyloxy)ethyl, 1-(ethoxycarbonyloxy)ethyl, ethoxycarbonyloxymethyl, 1-(isopropoxycarbonyloxy)ethyl, 1-(t-butoxycarbonyloxy)ethyl, 1-(ethoxycarbonyloxy)propyl, or 1-(cyclohexyloxycarbonyloxy)ethyl group; a phthalidyl group; a carbonyloxyalkyl group, for example, an oxodioxolenylmethyl group such as a 4-methyl-oxodioxolenylmethyl, 4-phenyl-oxodioxolenylmethyl, or oxodioxolenylmethyl group; the aliphatic acyl groups described hereinbefore; the aromatic acyl groups described hereinbefore; a residual group of a half ester of succinic acid; a residual group of an ester of phosphoric acid; a residual group of an ester formation of an amino acid; a carbamoyl group; and a carbamoyl group substituted with 1 or 2 lower alkyl groups. Whether a derivative prepared has such a group can be determined as follows. The derivative under investigation is intravenously administered to a test animal such as a rat or mouse and the body fluids of the test animal are thereafter studied. If the parent compound of said derivative or a pharmacologically acceptable salt of the parent compound is removed in said body fluid, said derivative under investigation is judged to have a protective group which can be deprotected by a biological process. Of these protective groups described above, a residual group of an ester of phosphoric acid is preferred.

The amino alcohol derivatives having the general formula (I) of the present invention, pharmacologically acceptable salts thereof and pharmacologically acceptable esters thereof have asymmetric carbon atoms in their structures and can exist as optical isomers. In the amino alcohol derivatives having the general formula (I), a single optical isomer and a mixture of optical isomers are represented by the single general formula (I). The present invention encompasses all the optical isomers and mixtures thereof in optional ratios. For example, in the amino alcohol derivatives having the general formula (I) of the present invention, the amino group is attached to an asymmetric carbon atom, and the particularly preferred absolute configuration at this asymmetric carbon is the R configuration.

When the amino alcohol derivatives having the general formula (I), pharmacologically acceptable salts thereof and pharmacologically acceptable esters thereof are allowed to stand so that they are exposed to the atmosphere or are recrystallized, they may absorb water or water may be attached to them to form a hydrate. Such hydrates are also encompassed as the pharmacologically acceptable salts of amino alcohol derivatives having the general formula (I) of the present invention.

The present invention includes the following compositions:

A pharmaceutical composition in which
R¹ and R² are each a hydrogen atom,
R³ is a methyl group, an ethyl group or a hydroxymethyl group,
R⁴ is a hydrogen atom or a halogen atom,
R⁵ is a hydrogen atom, an unsubstituted phenyl group or a phenyl group substituted with from 1 to 3 substituents from Substituent group a,
Z is a single bond or a C₁-C₈ alkylene group, and
n is an integer of 2.

A pharmaceutical composition in which the HMG-COA reductase inhibitor is atorvastatin and the amino alcohol compound having the formula (I) is
2-amino-2-methyl-4-{1-methyl-5-[4-(4-methylphenyl)butanoyl]pyrrol-2-yl}butan-1-ol.

A pharmaceutical composition in which the HMG-COA reductase inhibitor is atorvastatin and the amino alcohol compound having the formula (I) is
2-amino-2-methyl-4-{1-methyl-5-[4-(3,4-dimethylphenyl)butanoyl]pyrol-2-yl}butan-1-ol.

A pharmaceutical composition in which the HMG-COA reductase inhibitor is atorvastatin and the amino alcohol compound having the formula (I) is
2-amino-2-methyl-4-{1-methyl-5-[5-(4-methylphenyl)pentanoyl]pyrrol-2-yl}butan-1-ol.

A pharmaceutical composition in which the HMG-COA reductase inhibitor is atorvastatin and the amino alcohol compound having the formula (I) is
2-amino-2-methyl-4-{1-methyl-5-[5-(3,4-dimethylphenyl)pentanoyl]pyrrol-2-yl}butan-1-ol.

A pharmaceutical composition in which the HMG-COA reductase inhibitor is pravastatin and the amino alcohol compound having the formula (I) is 2-amino-2-methyl-4-{1-methyl-5-[4-(4-methylphenyl)butanoyl]pyrrol-2-yl}butan-1-ol.

A pharmaceutical composition in which the HMG-COA reductase inhibitor is pravastatin and the amino alcohol compound having the formula (I) is
2-amino-2-methyl-4-{1-methyl-5-[4-(3,4-dimethylphenyl)butanoyl]pyrrol-2-yl}butan-1-ol.

A pharmaceutical composition in which the HMG-COA reductase inhibitor is pravastatin and the amino alcohol compound having the formula (I) is 2-amino-2-methyl-4-{1-methyl-5-[5-(4-methylphenyl)pentanoly]pyrrol-2-yl}butan-1-ol.

A pharmaceutical composition in which the HMG-COA reductase inhibitor is pravastatin and the amino alcohol compound having the formula (I) is
2-amino-2-methyl-4-{1-methyl-5-[5-(3,4-dimethylphenyl)pentanoyl]pyrrol-2-yl}butan-1-ol.

A pharmaceutical composition in which the HMG-COA reductase inhibitor is pravastatin and the amino alcohol compound having the formula (I) is 2-amino-2-[2-(4-octylphenyl)ethyl]propan-1,3-diol.

Typical examples of compounds having the general formula (I) of the present invention are listed in the following Table 1, but the present invention should not be limited to these compounds.

The meaning of the abbreviations in the following Table is shown below.
Ac: acetyl group
tBu: t-butyl group
Et: ethyl group
EtO: ethoxy group
Me: methyl group
MeO: methoxy group
Ph: phenyl group
cPr: cyclopropyl group,

iPr: isopropyl group

TABLE 1

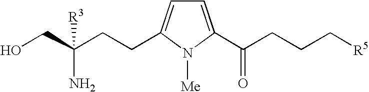

| Compound | | |
|---|---|---|
| No. | R³ | R⁵ |
| 1 | Me | 2-F-Ph |
| 2 | Me | 3-F-Ph |
| 3 | Me | 4-F-Ph |
| 4 | Me | 2,3-di-F-Ph |
| 5 | Me | 2,4-di-F-Ph |
| 6 | Me | 2,5-di-F-Ph |
| 7 | Me | 3,4-di-F-Ph |
| 8 | Me | 3,5-di-F-Ph |
| 9 | Me | 2-Cl-Ph |
| 10 | Me | 3-Cl-Ph |
| 11 | Me | 4-Cl-Ph |
| 12 | Me | 2,3-di-Cl-Ph |
| 13 | Me | 2,4-di-Cl-Ph |
| 14 | Me | 2,5-di-Cl-Ph |
| 15 | Me | 3,4-di-Cl-Ph |
| 16 | Me | 3,5-di-Cl-Ph |
| 17 | Me | 2-Me-Ph |
| 18 | Me | 3-Me-Ph |
| 19 | Me | 4-Me-Ph |
| 20 | Me | 2,3-di-Me-Ph |
| 21 | Me | 2,4-di-Me-Ph |
| 22 | Me | 2,5-di-Me-Ph |
| 23 | Me | 3,4-di-Me-Ph |
| 24 | Me | 3,5-di-Me-Ph |
| 25 | Me | 2-Et-Ph |
| 26 | Me | 3-Et-Ph |
| 27 | Me | 4-Et-Ph |
| 28 | Me | 2-cPr-Ph |
| 29 | Me | 3-cPr-Ph |
| 30 | Me | 4-cPr-Ph |
| 31 | Me | 2-iPr-Ph |
| 32 | Me | 3-iPr-Ph |
| 33 | Me | 4-iPr-Ph |
| 34 | Me | 2-tBu-Ph |
| 35 | Me | 3-tBu-Ph |
| 36 | Me | 4-tBu-Ph |
| 40 | Me | 2-MeO-Ph |
| 41 | Me | 3-MeO-Ph |
| 42 | Me | 4-MeO-Ph |
| 43 | Me | 2,3-di-MeO-Ph |
| 44 | Me | 2,4-di-MeO-Ph |
| 45 | Me | 2,5-di-MeO-Ph |
| 46 | Me | 3,4-di-MeO-Ph |
| 47 | Me | 3,5-di-MeO-Ph |
| 48 | Me | 2-EtO-Ph |
| 49 | Me | 3-EtO-Ph |
| 50 | Me | 4-EtO-Ph |
| 51 | Me | 2-iPrO-Ph |
| 52 | Me | 3-iPrO-Ph |
| 53 | Me | 4-iPrO-Ph |
| 54 | Me | 2-Me-3-MeO-Ph |
| 55 | Me | 2-Me-4-MeO-Ph |
| 56 | Me | 2-Me-5-MeO-Ph |
| 57 | Me | 3-Me-4-MeO-Ph |
| 58 | Me | 3-Me-5-MeO-Ph |
| 59 | Me | 2-MeO-3-Me-Ph |
| 60 | Me | 2-MeO-4-Me-Ph |
| 61 | Me | 2-MeO-5-Me-Ph |
| 62 | Me | 3-MeO-4-Me-Ph |
| 63 | Me | 3-MeO-5-Me-Ph |
| 64 | Me | 2-CF₃-Ph |
| 65 | Me | 3-CF₃-Ph |
| 66 | Me | 4-CF₃-Ph |
| 67 | Me | 3,5-di-CF₃-Ph |
| 68 | Me | 2-Ac-Ph |
| 69 | Me | 3-Ac-Ph |
| 70 | Me | 4-Ac-Ph |
| 71 | Me | 2-CN-Ph |
| 72 | Me | 3-CN-Ph |
| 73 | Me | 4-CN-Ph |
| 74 | Et | 2-F-Ph |
| 75 | Et | 3-F-Ph |
| 76 | Et | 4-F-Ph |
| 77 | Et | 2,3-di-F-Ph |
| 78 | Et | 2,4-di-F-Ph |
| 79 | Et | 2,5-di-F-Ph |
| 80 | Et | 3,4-di-F-Ph |
| 81 | Et | 3,5-di-F-Ph |
| 82 | Et | 2-Cl-Ph |
| 83 | Et | 3-Cl-Ph |
| 84 | Et | 4-Cl-Ph |
| 85 | Et | 2,3-di-Cl-Ph |
| 86 | Et | 2,4-di-Cl-Ph |
| 87 | Et | 2,5-di-Cl-Ph |
| 88 | Et | 3,4-di-Cl-Ph |
| 89 | Et | 3,5-di-Cl-Ph |
| 90 | Et | 2-Me-Ph |
| 91 | Et | 3-Me-Ph |
| 92 | Et | 4-Me-Ph |
| 93 | Et | 2,3-di-Me-Ph |
| 94 | Et | 2,4-di-Me-Ph |
| 95 | Et | 2,5-di-Me-Ph |
| 96 | Et | 3,4-di-Me-Ph |
| 97 | Et | 3,5-di-Me-Ph |
| 98 | Et | 2-Et-Ph |
| 99 | Et | 3-Et-Ph |
| 100 | Et | 4-Et-Ph |
| 101 | Et | 2-cPr-Ph |
| 102 | Et | 3-cpr-Ph |
| 103 | Et | 4-cPr-Ph |
| 104 | Et | 2-iPr-Ph |
| 105 | Et | 3-iPr-Ph |
| 106 | Et | 4-iPr-Ph |
| 107 | Et | 2-tBu-Ph |
| 108 | Et | 3-tBu-Ph |
| 109 | Et | 4-tBu-Ph |
| 113 | Et | 2-MeO-Ph |
| 114 | Et | 3-MeO-Ph |
| 115 | Et | 4-MeO-Ph |
| 116 | Et | 2,3-di-MeO-Ph |
| 117 | Et | 2,4-di-MeO-Ph |
| 118 | Et | 2,5-di-MeO-Ph |
| 119 | Et | 3,4-di-MeO-Ph |
| 120 | Et | 3,5-di-MeO-Ph |
| 121 | Et | 2-EtO-Ph |
| 122 | Et | 3-EtO-Ph |
| 123 | Et | 4-EtO-Ph |
| 124 | Et | 2-iPrO-Ph |
| 125 | Et | 3-iPrO-Ph |
| 126 | Et | 4-iPrO-Ph |
| 127 | Et | 2-Me-3-MeO-Ph |
| 128 | Et | 2-Me-4-MeO-Ph |
| 129 | Et | 2-Me-5-MeO-Ph |
| 130 | Et | 3-Me-4-MeO-Ph |
| 131 | Et | 3-Me-5-MeO-Ph |
| 132 | Et | 2-MeO-3-Me-Ph |
| 133 | Et | 2-MeO-4-Me-Ph |
| 134 | Et | 2-MeO-5-Me-Ph |
| 135 | Et | 3-MeO-4-Me-Ph |
| 136 | Et | 3-MeO-5-Me-Ph |
| 137 | Et | 2-CF₃-Ph |
| 138 | Et | 3-CF₃-Ph |
| 139 | Et | 4-CF₃-Ph |
| 140 | Et | 3,5-di-CF₃-Ph |
| 141 | Et | 2-Ac-Ph |
| 142 | Et | 3-Ac-Ph |
| 143 | Et | 4-Ac-Ph |
| 144 | Et | 2-CN-Ph |
| 145 | Et | 3-CN-Ph |
| 146 | Et | 4-CN-Ph |

In Table 1 described above, the preferred compounds (I) of the present invention include
Exemplification compound numbers: 17-24, 40-47, 54-63, 72, 73, 90-97, 113-120, 127-136, 145 and 146, and the more preferred compounds include
2-amino-2-methyl-4-{1-methyl-5-[4-(2-methylphenyl)butanoyl]pyrrol-2-yl}butan-1-ol,
2-amino-2-methyl-4-{1-methyl-5-[4-(3-methylphenyl)butanoyl]pyrrol-2-yl}butan-1-ol,
2-amino-2-methyl-4-{1-methyl-5-[4-(4-methylphenyl)butanoyl]pyrrol-2-yl}butan-1-ol,
2-amino-2-methyl-4-{1-methyl-5-[4-(2,3-dimethylphenyl)butanoyl]pyrrol-2-yl}butan-1-ol,
2-amino-2-methyl-4-{1-methyl-5-[4-(2,4-dimethylphenyl)butanoyl]pyrrol-2-yl}butan-1-ol,
2-amino-2-methyl-4-{1-methyl-5-[4-(2,5-dimethylphenyl)butanoyl]pyrrol-2-yl}butan-1-ol,
2-amino-2-methyl-4-{1-methyl-5-[4-(3,4-dimethylphenyl)butanoyl]pyrrol-2-yl}butan-1-ol,
2-amino-2-methyl-4-{1-methyl-5-[4-(3,5-dimethylphenyl)butanoyl]pyrrol-2-yl}butan-1-ol,
2-amino-2-methyl-4-{1-methyl-5-[4-(4-ethylphenyl)butanoyl]pyrrol-2-yl}butan-1-ol,
2-amino-2-methyl-4-{1-methyl-5-[4-(4-isopropylphenyl)butanoyl]pyrrol-2-yl}butan-1-ol,
2-amino-2-methyl-4-{1-methyl-5-[4-(4-cyclopropylphenyl)butanoyl]pyrrol-2-yl}butan-1-ol,
2-amino-2-methyl-4-{1-methyl-5-[4-(4-t-butylphenyl)butanoyl]pyrrol-2-yl}butan-1-ol,
2-amino-2-methyl-4-{1-methyl-5-[4-(4-methoxyphenyl)butanoyl]pyrrol-2-yl}butan-1-ol,
2-amino-2-methyl-4-{1-methyl-5-[4-(3,4-dimethoxyphenyl)butanoyl]pyrrol-2-yl}butan-1-ol,
2-amino-2-methyl-4-{1-methyl-5-[4-(3-methyl-4-methoxyphenyl) butanoyl]pyrrol-2-yl}butan-1-ol,
2-amino-2-methyl-4-{1-methyl-5-[4-(3-methoxy-4-methylphenyl) butanoyl]pyrrol-2-yl}butan-1-ol,
2-amino-2-methyl-4-{1-methyl-5-[4-(4-fluorophenyl)butanoyl]pyrrol-2-yl}butan-1-ol,
2-amino-2-methyl-4-{1-methyl-5-[4-(4-chlorophenyl)butanoyl]pyrrol-2-yl}butan-1-ol,
2-amino-2-methyl-4-{1-methyl-5-[4-(4-trifluoromethylphenyl) butanoyl]pyrrol-2-yl}butan-1-ol,
2-amino-2-methyl-4-{1-methyl-5-[4-(4-cyanophenyl)butanoyl]pyrrol-2-yl}butan-1-ol,
2-amino-2-ethyl-4-{1-methyl-5-[4-(2-methylphenyl)butanoyl]pyrrol-2-yl}butan-1-ol,
2-amino-2-ethyl-4-{1-methyl-5-[4-(3-methylphenyl)butanoyl]pyrrol-2-yl}butan-1-ol,
2-amino-2-ethyl-4-{1-methyl-5-[4-(4-methylphenyl)butanoyl]pyrrol-2-yl}butan-1-ol,
2-amino-2-ethyl-4-{1-methyl-5-[4-(2,3-dimethylphenyl)butanoyl]pyrrol-2-yl}butan-1-ol,
2-amino-2-ethyl-4-{1-methyl-5-[4-(2,4-dimethylphenyl)butanoyl]pyrrol-2-yl}butan-1-ol,
2-amino-2-ethyl-4-{1-methyl-5-[4-(2,5-dimethylphenyl)butanoyl]pyrrol-2-yl}butan-1-ol,
2-amino-2-ethyl-4-{1-methyl-5-[4-(3,4-dimethylphenyl)butanoyl]pyrrol-2-yl}butan-1-ol,
2-amino-2-ethyl-4-{1-methyl-5-[4-(3,5-dimethylphenyl)butanoyl]pyrrol-2-yl}butan-1-ol,
2-amino-2-ethyl-4-{1-methyl-5-[4-(4-ethylphenyl)butanoyl]pyrrol-2-yl}butan-1-ol,
2-amino-2-ethyl-4-{1-methyl-5-[4-(4-isopropylphenyl)butanoyl]pyrrol-2-yl}butan-1-ol,
2-amino-2-ethyl-4-{1-methyl-5-[4-(4-cyclopropylphenyl)butanoyl]pyrrol-2-yl}butan-1-ol,
2-amino-2-ethyl-4-{1-methyl-5-[4-(4-t-butylphenyl)butanoyl]pyrrol-2-yl}butan-1-ol,
2-amino-2-ethyl-4-{1-methyl-5-[4-(4-methoxyphenyl)butanoyl]pyrrol-2-yl}butan-1-ol,
2-amino-2-ethyl-4-{1-methyl-5-[4-(3,4-dimethoxyphenyl)butanoyl]pyrrol-2-yl}butan-1-ol,
2-amino-2-ethyl-4-{1-methyl-5-[4-(3-methyl-4-methoxyphenyl) butanoyl]pyrrol-2-yl}butan-1-ol,
2-amino-2-ethyl-4-{1-methyl-5-[4-(4-fluorophenyl)butanoyl]pyrrol-2-yl}butan-1-ol,
2-amino-2-ethyl-4-{1-methyl-5-[4-(4-chlorophenyl)butanoyl]pyrrol-2-yl}butan-1-ol,
2-amino-2-ethyl-4-{1-methyl-5-[4-(4-trifluoromethylphenyl) butanoyl]pyrrol-2-yl}butan-1-ol,
2-amino-2-ethyl-4-{1-methyl-5-[4-(4-cyanophenyl)butanoyl]pyrrol-2-yl}butan-1-ol,
   the still more preferred compounds include Exemplification compound numbers: 17-24, 54-63, 72 and 73, and the most preferred compounds include
Exemplification compound number 17:
2-amino-2-methyl-4-{1-methyl-5-[4-(2-methylphenyl)butanoyl]pyrrol-2-yl}butan-1-ol,
Exemplification compound number 18:
2-amino-2-methyl-4-{1-methyl-5-[4-(3-methylphenyl)butanoyl]pyrrol-2-yl}butan-1-ol,
Exemplification compound number 19:
2-amino-2-methyl-4-{1-methyl-5-[4-(4-methylphenyl)butanoyl]pyrrol-2-yl}butan-1-ol,
Exemplification compound number 20:
2-amino-2-methyl-4-{1-methyl-5-[4-(2-methylphenyl)butanoyl]pyrrol-2-yl}butan-1-ol,
Exemplification compound number 21:
2-amino-2-methyl-4-{1-methyl-5-[4-(2,4-dimethylphenyl)butanoyl]pyrrol-2-yl}butan-1-ol,
Exemplification compound number 22:
2-amino-2-methyl-4-{1-methyl-5-[4-(2,5-dimethylphenyl)butanoyl]pyrrol-2-yl}butan-1-ol,
Exemplification compound number 23:
2-amino-2-methyl-4-{1-methyl-5-[4-(3,4-dimethylphenyl)butanoyl]pyrrol-2-yl}butan-1-ol,
Exemplification compound number 24:
2-amino-2-methyl-4-{1-methyl-5-[4-(3,5-dimethylphenyl)butanoyl]pyrrol-2-yl}butan-1-ol,
Exemplification compound number 57:
2-amino-2-methyl-4-{1-methyl-5-[4-(3-methyl-4-methoxyphenyl)butanoyl]pyrrol-2-yl}butan-1-ol,
Exemplification compound number 62:
2-amino-2-methyl-4-{1-methyl-5-[4-(3-methoxy-4-methylphenyl)butanoyl]pyrrol-2-yl}butan-1-ol, and
Exemplification compound number 73:
2-amino-2-methyl-4-{1-methyl-5-[4-(4-cyanophenyl)butanoyl]pyrrol-2-yl}butan-1-ol.

Since medicinal compositions of the present invention exert excellent immunosuppressing effects with low toxicity, the medical compositions of the present invention are useful as prophylactic or therapeutic agents for immune-related diseases such as reject reactions caused by organ or skin transplantation, rheumatic arthritis, and other autoimmune diseases in mammals (particularly in humans).

HMG-COA reductase inhibitors used as an active ingredient in the medicinal composition of the present invention can easily be prepared according to the methods from Patent literature 8 to Patent literature 14 described hereinbefore (Japanese Patent Publication (Kokai) Number Sho 57-2240 (U.S. Pat. No. 4,346,227) , Japanese Patent Publication (Kokai) Number Sho 57-163374 (U.S. Pat. No. 4,231,938), Japanese Patent Publication (Kokai) Number Sho 56-122375 (U.S. Pat. No. 4,444,784), Japanese Patent Publication (Kohyo) Number Sho 60-500015 (U.S. Pat. No. 4,739,073), Japanese Patent Publication (Kokai) Number Hei 3-58967 (U.S. Pat. No. 5,273,995), Japanese Patent Publication (Kokai) Number Hei 5-178841 (U.S. Pat. No. 5,260,440), Japanese Patent Publication (Kokai) Number Hei 1-279866 (U.S. Pat. No. 5,854,259 and U.S. Pat. No. 5,856,336)), or the like.

In addition, the amino alcohol derivatives, which are one of the active ingredients of the medicinal composition of the present invention, can easily be prepared, for example, according to the methods from Patent literature 1 to Patent literature 7 described hereinbefore (WO 94/08943 (EP627406), W096/06068, W098/45249, WO03/029184, WO03/029205, Japanese Patent Publication (Kokai) Number 2002-167382, Japanese Patent Publication (Kokai) Number2003-267950) and the like.

The medicinal compositions of the present invention which contain both an HMG-COA reductase inhibitor and an amino alcohol derivative augment the pharmacological immunosuppressive activity and lower its side effect and toxicity. Thus the medicinal composition of the present invention is useful as a prophylactic or therapeutic agent for autoimmune diseases such as systemic lupus erythematosus, rheumatoid arthritis, polymyositis, dermatomyositis, scleroderma, Behcet's syndrome, Crohn's disease, ulcerative colitis, autoimmune hepatitis, a plastic anemia, idiopathic thrombocytopenic purpura, autoimmune hemolytic anemia, multiple sclerosis, autoimmunebullosis, psoriasis vulgaris, vasculitis syndrome, Wegener's granuloma, uveitis, idiopathic interstitial pneumonia, Goodpasture's syndrome, sarcoidosis, allergic granulomatous angitis, bronchial asthma, myocarditis, cardiomyopathy, aortitis syndrome, postmyocardial infarction syndrome, primary pulmonary hypertension, minimal change nephrotic syndrome, membranous nephropathy, membranoproliferative glomerulonephritis, focal glomerular sclerosis, crescentic glomerulonephritis, myasthenia gravis, inflammatory neuropathy, atopic dermatitis, chronic actinic dermatitis, acute polyarthritis, Sydenham's chorea, systemic sclerosis, adult-onset type diabetes mellitus, insulin dependent diabetes mellitus, juvenile diabetes mellitus, atherosclerosis, glomerularnephritis, tubulointerstitial nephritis, primary biliary cirrhosis, primary sclerosing cholangitis, fulminant hepatitis, viral hepatitis, GVHD, reject reactions caused by transplantation of various organs, contact dermatitis, and sepsis, or other immunology-related diseases.

In the case that the medicinal composition of the present invention is used as a prophylactic or therapeutic agent for diseases described above, the medicinal composition of the present invention can be administered in a suitable dosage form by mixing with a suitable pharmacologically acceptable excipient and/or diluent, for example, as tablets, capsules, granules, powders, or syrups for oral administration, or injections or suppositories for parenteral administration.

The medicinal compositions are prepared, according to well known techniques, using additives such as excipients (for example, organic excipients including sugar derivatives such as lactose, sucrose, glucose, mannitol, and sorbitol; starch derivatives such as corn starch, potato starch, a-starch, and dextrin; cellulose derivatives such as crystalline cellulose; gum arabic; dextran; pullulan; and inorganic excipients including silicate derivatives such as light anhydrous silicic acid, synthetic aluminum silicate, calcium silicate, and magnesium aluminometasilicate; phosphates such as calcium hydrogenphosphate; carbonates such as calcium carbonate; and sulfates such as calcium sulfate can be listed), lubricants (for example, stearic acid and metal salts of stearic acid such as calcium stearate and magnesium stearate; talc; colloidal silica; waxes such as veegum and spermaceti; boric acid; adipic acid; sulfates such as sodium sulfate; glycol; fumaric acid; sodium benzoate; DL-leucine; sodium fatty acid; lauryl sulfates such as sodium lauryl sulfate and magnesium lauryl sulfate; silicates such as silicic anhydride and silicic hydrate; and the starch derivatives described above can be listed), binders (for example, hydroxypropylcellulose, hydroxypropylmethylcellulose, polyvinylpyrrolidone, Macrogol, and similar excipients to those described above can be listed), disintegrants (for example, cellulose derivatives such as low-substituted hydroxypropylcellulose, carboxymethylcellulose, calcium carboxymethylcellulose, and internally crosslinked sodium carboxymethylcellulose; and chemically modified starch/cellulose derivatives such as carboxymethylstarch, sodium carboxymethylstarch, and crosslinked polyvinylpyrrolidone can be listed), stabilizers (for example, paraoxybenzoates such as methylparaben and propylparaben; alcohols such as chlorobutanol, benzyl alcohol, and phenylethyl alcohol; benzalkonium chloride; phenols such as phenol and cresol; thimerosal; dehydroacetic acid; and sorbic acid can be listed), flavourings (for example, conventionally employed sweeteners, acidifiers, and flavourings can be listed), diluents, and the like.

In the administration of the medicinal composition of the present invention, at least one HMG-COA reductase inhibitor selected from the specified group described hereinbefore and at least one compound selected from the group consisting of amino alcohol derivatives having the general formula (I) described hereinbefore, pharmacologically acceptable salts thereof and pharmacologically acceptable esters thereof can be administered simultaneously, sequentially or separately on different occasions. In clinical practice, however, it is convenient to administer these drugs at the same time, and accordingly, said HMG-COA reductase inhibitor and at least one compound selected from the group consisting of amino alcohol derivatives having the general formula (I) described hereinbefore, pharmacologically acceptable salts thereof and pharmacologically acceptable esters thereof can be administered in the form of a combination drug.

When it is undesirable to mix the pharmaceutical preparations of both drugs together physically, it is possible to administer these drugs simultaneously as a kit comprising the pharmaceutical preparations of each drug, and it is also possible to administer each of the pharmaceutical preparations of these drugs one after the other at a suitable interval, since these drugs exert a prominent effect even if these drugs are not administered simultaneously.

In the present invention, a simultaneous administration is not particularly restricted provided that these drugs have a pharmaceutical dose form that can be administered at almost the same time, but it is preferable to administer them as a single medicinal composition.

In the present invention, sequential or separate administration is not particularly restricted provided that these drugs have a pharmaceutical dose form that can be administered separately at different times, and means, for example, that at least one compound selected from the group consisting of amino alcohol derivatives having the general formula (I) described hereinbefore, pharmacologically acceptable salts thereof and pharmacologically acceptable esters thereof is administered first and then at least one HMG-COA reductase inhibitor selected from the specified group described hereinbefore is administered after a defined time, or that said HMG-COA reductase inhibitor is administered first and then at least one compound selected from the group consisting of amino alcohol derivatives having the general formula (I) described hereinbefore, pharmacologically acceptable salts thereof and pharmacologically acceptable esters thereof is administered after a defined time. In the present invention, said HMG-COA reductase inhibitor and at least one compound selected from the group consisting of amino alcohol derivatives having the general formula (I) described hereinbefore, pharmacologically acceptable salts thereof and pharmacologically acceptable esters thereof can be administered separately, and the maximum time interval, between administration of these drugs, acceptable for achieving said prominent effect by these drugs can be confirmed in clinical practice or by animal experiments.

Each dose of at least one HMG-COA reductase inhibitor selected from the specified group described hereinbefore and at least one compound selected from the group consisting of amino alcohol derivatives having the general formula (I) described hereinbefore, pharmacologically acceptable salts thereof and pharmacologically acceptable esters thereof, both of which are active ingredients of the present invention, and the dose ratio between these drugs, may vary depending on a variety of factors such as the activity of each drug, and the symptoms, age and body weight of the patient.

The dose of the combination of at least one agent selected from HMG-COA reductase inhibitors defined hereinbefore and at least one compound selected from the group consisting of the amino alcohol derivatives having the general formula (I) described hereinbefore, pharmacologically acceptable salts thereof or pharmacologically acceptable esters thereof may vary depending on a variety of factors such as the symptoms and age of the patient. For example, in the case of oral administration, the dose is between 0.05 mg and 200 mg per one time for a human adult (body weight: about 60 kg), and is preferably between 5 mg and 40 mg. In the case of intravenous administration, the dose is between 0.01 mg and 100 mg per one time for a human adult (body weight: about 60 kg), and is preferably between 1 mg and 10 mg. The dosing frequency is from one to six times per day for a human adult (body weight: about 60 kg), and the defined daily dosage may be administered throughout the day at the same time or at certain intervals depending on the symptoms of the patient.

The dose ratio between at least one agent selected from HMG-COA reductase inhibitors defined hereinbefore and at least one compound selected from the group consisting of the amino alcohol derivatives having the general formula (I) described hereinbefore, pharmacologically acceptable salts thereof and pharmacologically acceptable esters thereof, both of which are active ingredients of medicinal compositions of the present invention, may also vary considerably, but the dose ratio by weight of said HMG-COA reductase inhibitor to at least one compound selected from the group of the amino alcohol derivatives having the general formula (I) described hereinbefore, pharmacologically acceptable salts thereof or pharmacologically acceptable esters thereof may be within a range of 5:1-10000:1, preferably 5:1-6600:1, more preferably 5:1-2000:1, and particularly preferably 400:1-2000:1.

### EXAMPLES

The present invention will hereinafter be described in more detail by way of the Examples, Test Examples, and Formulation Examples below, but the scope of the present invention should not be limited to these examples.

### Test Example 1

Determination of Inhibitory Activities of the Compound against Host versus Graft Reaction in the Rat

(1) Two strains of rats [Lewis rats (male, 6 weeks of age, Charles River Japan Inc.) and WKAH/Hkm rats (male, 7 weeks of age, Japan SLC Inc.) are used. Five rats (host) per group are used.
   (2) Induction of HvGR

Spleen cells are isolated from the spleens of WKAH/Hkm or Lewis rats and floated in RPMI1640 medium (Life Technologies Inc.) at a concentration of 1×10⁸ cells/ml. 0.1 ml of the medium containing the free-floating spleen cells of WKAH/Hkm rats is then intracutaneously injected into the bilateral foot-pads of the hind limbs of both sides of Lewis rats (HvGR induction group) or that of Lewis rats is then intracutaneously injected into the bilateral foot-pads of the hind limbs of both sides of Lewis rats (the same strain group).
(3) Administration of the Compound

The test compound is suspended in 0.5% tragacantha solution at concentrations of 0.8 mg/5 ml, 0.08 mg/5 ml, and 0.008 mg/5 ml.

In groups of rats administered with a statin and an immunosuppressant, a suspended solution of a statin and an immunosuppressant is orally administered at a volume of 5 ml/kg, once daily, for 4 successive days starting on the day of the spleen cell injection.

In addition, a statin suspended solution and tragacantha solution (0.5%) is orally administered to rats in a “statin administration group”, while an immunosuppressant and tragacantha solution (0.5%) is orally administered to rats in an “immunosuppressant administration group”. The administration volumes in the rats of both of these 2 groups are 5 ml/kg, once daily, for 4 successive days starting on the day of the spleen cell injection.

Furthermore, tragacantha solution (0.5%) is orally administered to rats in the “same strain group” (Lewis rats injected with spleen cells of Lewis rats, but not treated with the compound) and the control group (Lewis rats injected with spleen cells of WKAH/Hkm rats and not treated with the compound).

(4) Determination of Inhibitory Activity against HvGR

The average weight of the popliteal lymph nodes of the same strain rats is subtracted from the individual weights of the popliteal lymph nodes of individual rats (“Weight of the popliteal lymph nodes after HvGR-induction”). The inhibitory activities of compounds are calculated from the “Weight of the popliteal lymph nodes after HvGR-induction” of individual rats in the drug-treated group versus the average “Weight of the popliteal lymph nodes after HvGR-induction” in the control group (Equation 1):

$$Inhibitory Rate ( \% ) = \frac{\begin{matrix}\begin{matrix}\begin{matrix}( Average “ Weight of the popliteal lymph \\ nodes after HvGr \text{-} induction ” in \end{matrix} \\ control group ) - ( Average “ Weight of\end{matrix} \\ the popliteal lymph nodes after \\ HvGr \text{-} induction ” in drug \text{-} treated \\ group\end{matrix}}{\begin{matrix}( Average “ Weight of the popliteal lymph \\ nodes after HvGr \text{-} induction ” \\ in control group )\end{matrix}} \times 100$$

### Test Example 2

Arthritis Development Preventative Activity

The inhibitory activity of the medicinal composition of the present invention against arthritis is assessed by using the rate of inhibition of swelling of the right hindpaw as an indicator in an adjuvant-induced arthritis model which exhibits symptoms similar to human arthritis. Female Lewis rats aged 8 weeks are used in this study.

(1) Preparation of Adjuvant

Heat-killed dried Mycobacterium butyricum is ground on an agate mortar, and is then suspended in dry-sterilized liquid paraffin to make a 2 mg/ml suspension. The resulting suspended solution is then sonicated and used as adjuvant.

(2) Preparation of Compounds

Compounds are dissolved or suspended in 0.5% tragacantha solution.

(3) Induction of Adjuvant-Induced Arthritis

Arthritis is induced by intradermal injection of the adjuvant prepared in (1) described above into the heel of the right hind paw of rats in the drug-treated group and in the control group. Five rats per group are used. Non-treated rats are separately used as a normal control group.

(4) Administration of the Compound

The test compounds prepared in (2) described above are orally administered to the rats of the compound-treated group at a volume of 5 ml/kg, once a day from the injection day of the adjuvant (day 0) for 18 successive days. 0.5% tragacantha solution alone is similarly administered to rats in the control groups.

(5) Calculation of Inhibition Rate of Swollen Foot Volume by the Test Compound

On the 11th and 18th day after the drug administration is started, the right foot volume of each rat is measured by an apparatus for determination of the volume. The average swelling volume of each group is calculated. The percent inhibition of the swelling of the injected foot of the treated animals as compared to that of the control animals is calculated according to the following equation:
Inhibition rate of swollen foot volume (%)={1−[(swollen foot volume of compound-treated animals)−(swollen foot volume of normal control animals)]/[(swollen foot volume of control animals)−(swollen foot volume of normal control animals)]}×100

### Test Example 3

Assessment of Inhibitory Activity against Peripheral Lymphocytes of the Rat

Lewis rats (male, 5 weeks of age, Charles River Japan Inc.) are used. Five rats per group are used.

(1) Administration of the Compound

The test compound is suspended in 1% tragacantha solution (vehicle) The suspended solution of the test compound is orally administered to rats at a volume of 5 ml/kg.

In control rats, vehicle is orally administered, instead of the suspended solution of the compound.

(2) Counting of Peripheral Lymphocytes

3 hours after administration of either the vehicle or the suspended solution of the test compound, blood is collected from the postcaval vein of the rats under ether anesthesia. Then the collected blood is transferred into a tube containing EDTA. The absolute number of lymphocytes in the blood is counted using a full blood count analyser. The inhibitory activity (%) of the test compound is determined by calculation of the relative number of peripheral lymphocytes with the number of lymphocytes from normal rats being defined as 100%.

### Formulation Examples

Tablets

| | |
|---|---|
| A statin | 50.0 mg |
| An immunosuppressant | 10.0 mg |
| Lactose | 113.0 mg |
| Corn starch | 25.0 mg |
| Magnesium stearate | 2.0 mg |
| | 200.0 mg |

Tablets (200 mg in a tablet) are prepared by mixing powders of the above prescription in a blender, and a tableting the mixture using a tableting machine.

What is claimed is:

1. A pharmaceutical composition comprising a pharmaceutically effective amount of a combination of (i) at least HMG-CoA reductase inhibitor and (ii) at least amino alcohol compound having the following formula (I): 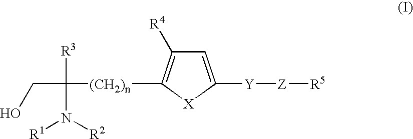
a pharmacologically acceptable salt thereof or a pharmacologically acceptable ester thereof, wherein
R¹ and R² are the same or different and each represents a hydrogen atom or a lower alkyl group,
R³ represents a lower alkyl group or a hydroxymethyl group,
R⁴ represents a hydrogen atom, a lower alkyl group, a lower alkoxy group or a halogen atom,
R⁵ represents a hydrogen atom, a halogen atom, a cyclohexyl group, an unsubstituted phenyl group or a phenyl group substituted with from 1 to 3 substituents from a Substituent group a,
X represents a vinylene group (CH=CH group), an oxygen atom, a sulfur atom or a methylated nitrogen atom,
Y represents a single bond, an oxygen atom, a sulfur atom or a carbonyl group,
Z represents a single bond, an unsubstituted C₁-C₈ alkylene group or a C₁-C₈ alkylene group substituted with from 2 to 8 fluorine atoms,
n represents an integer of 2 or 3,
Substituent group a is selected from the group consisting of a halogen atom, a cyano group, a lower alkyl group, a lower cycloalkyl group, a lower alkoxy group, a trifluoromethyl group, a phenyl group and a benzyloxy group.

2. A pharmaceutical composition according to claim 1, in which said amino alcohol compound having the formula (I) is a compound having the formula (Ia) shown below

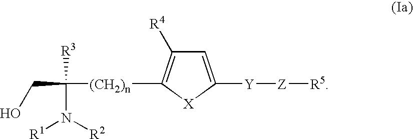

3. A pharmaceutical composition according to claim 1, in which the HMG-COA reductase inhibitor is at least one compound selected from the group consisting of pravastatin, lovastatin, simvastatin, fluvastatin, atorvastatin, rosvastatin and pivastatin.

4. A pharmaceutical composition according to claim 1, in which the HMG-COA reductase inhibitor is pravastatin.

5. A pharmaceutical composition according to claim 1, in which the HMG-COA reductase inhibitor is atorvastatin.

6. A pharmaceutical composition according to claim 1, in which R¹ and R² are a hydrogen atom.

7. A pharmaceutical composition according to claim 1, in which R³ is a methyl group, an ethyl group or a hydroxymethyl group.

8. A pharmaceutical composition according to claim 1, in which R³ is a hydroxymethyl group.

9. A pharmaceutical composition according to claim 1, in which R³ is a methyl group.

10. A pharmaceutical composition according to claim 1, in which R⁴ is a hydrogen atom.

11. A pharmaceutical composition according to claim 1, in which R⁵ is a phenyl group substituted with from 1 to 3 substituents selected from the group consisting of a fluorine atom, a cyano group, a methyl group, a methoxy group, an ethoxy group and a phenyl group.

12. A pharmaceutical composition according to claim 1, in which R⁵ is a phenyl group substituted with from 1 to 3 substituents selected from the group consisting of a methyl group and a methoxy group.

13. A pharmaceutical composition according to claim 1, in which R⁵ is a phenyl group.

14. A pharmaceutical composition according to claim 1, in which X is a vinylene group (CH═CH group).

15. A pharmaceutical composition according to claim 1, in which X is an oxygen atom, a sulfur atom or a nitrogen atom substituted with a methyl group.

16. A pharmaceutical composition according to claim 1, in which Y represents a carbonyl group.

17. A pharmaceutical composition according to claim 1, in which Z represents a single bond or a C₁-C₈ alkylene group.

18. A pharmaceutical composition according to claim 1, in which n represents an integer of 2.

19. A pharmaceutical composition according to claim 1, in which the amino alcohol compound having the formula (I) is selected from the group consisting of
2-amino-2-methyl-4-{1-methyl-5-[4-(2-methylphenyl)butanoyl]pyrrol-2-yl}butan-1-ol,
2-amino-2-methyl-4-{1-methyl-5-[4-(3-methylphenyl)butanoyl]pyrrol-2-yl}butan-1-ol,
2-amino-2-methyl-4-{1-methyl-5-[4-(4-methylphenyl)butanoyl]pyrrol-2-yl}butan-1-ol,
2-amino-2-methyl-4-{1-methyl-5-[4-(2,3-dimethylphenyl)butanoyl]pyrrol-2-yl}butan-1-ol,
2-amino-2-methyl-4-{1-methyl-5-[4-(2,4-dimethylphenyl)butanoyl]pyrrol-2-yl}butan-1-ol,
2-amino-2-methyl-4-{1-methyl-5-[4-(2,5-dimethylphenyl)butanoyl]pyrrol-2-y})butan-1-ol,
2-amino-2-methyl-4-{1-methyl-5-[4-(3,4-dimethylphenyl)butanoyl]pyrrol-2-yl}butan-1-ol,
2-amino-2-methyl-4-{1-methyl-5-[4-(3,5-dimethylphenyl)butanoyl]pyrrol-2-yl}butan-1-ol,
2-amino-2-methyl-4-{1-methyl-5-[4-(3-methyl-4-methoxyphenyl) butanoyl]pyrrol-2-yl}butan-1-ol,
2-amino-2-methyl-4-{1-methyl-5-[4-(3-methoxy-4-methylphenyl) butanoyl]pyrrol-2-yl}butan-1-ol,
2-amino-2-methyl-4-{1-methyl-5-(4-(4-cyanophenyl)butanoyl]pyrrol-2-yl}butan-1-ol,
2-amino-2-methyl-4-{1-methyl-5-[5-(2-methylphenyl)pentanoyl]pyrrol-2-yl}butan-1-ol,
2-amino-2-methyl-4-{1-methyl-5-[5-(3-methylphenyl)pentanoyl]pyrrol-2-yl}butan-1-ol,
2-amino-2-methyl-4-{1-methyl-5-[5-(4-methylphenyl)pentanoyl]pyrrol-2-yl}butan-1-ol,
2-amino-2-methyl-4-{1-methyl-5-[5-(2,3-dimethylphenyl)pentanoyl]pyrrol-2-yl}butan-1-ol,
2-amino-2-methyl-4-{1-methyl-5-[5-(2,4-dimethylphenyl)pentanoyl]pyrrol-2-yl}butan-1-ol,
2-amino-2-methyl-4-{1-methyl-5-[5-(2,5-dimethylphenyl)pentanoyl]pyrrol-2-yl}butan-1-ol,
2-amino-2-methyl-4-{1-methyl-5-[5-(3,4-dimethylphenyl)pentanoyl]pyrrol-2-yl}butan-1-ol,
2-amino-2-methyl-4-{1-methyl-5-[5-(3,5-dimethylphenyl)pentanoyl]pyrrol-2-yl}butan-1-ol,
2-amino-2-methyl-4-{1-methyl-5-[5-(3-methyl-4-methoxyphenyl) pentanoyl]pyrrol-2-yl}butan-1-ol,
2-amino-2-methyl-4-{1-methyl-5-[5-(3-methoxy-4-methylphenyl) pentanoyl]pyrrol-2-yl}butan-1-ol and
2-amino-2-methyl-4-{1-methyl-5-[5-(4-cyanophenyl)pentanoyl]pyrrol-2-yl}butan-1-ol.

20. A pharmaceutical composition according to claim 1, in which the amino alcohol compound having the formula (I) is selected from the group consisting of
2-amino-2-[2-(4-octylphenyl)ethyl]propan-1,3-diol,
2-amino-2-[2-(4-heptylphenyl)ethyl]propan-1,3-diol,
2-amino-2-{2-[4-(5-phenylpentanoyl)phenyl]ethyl}propan-1,3-diol,
2-amino-2-{2-[4-(5-cyclohexylpentanoyl)phenyl]ethyl}propan-1,3-diol,
2-amino-2-{2-[4-(7-phenylheptanoyl)phenyl]ethyl}propan-1,3-diol,
2-amino-2-[2-(4-[2-(4-methoxyphenyl)ethoxy]phenyl)ethyl]propan-1,3-diol,
2-amino-2-[2-(4-[2-(4-ethoxyphenyl)ethoxy]phenyl)ethyl]propan-1,3-diol,
2-amino-2-[2-(4-[2-(3-fluoro-4-methoxyphenyl)ethoxy]phenyl) ethyl]propan-1,3-diol,
2-amino-2-methyl-4-[4-(4,4,5,5,5-pentafluoropentoxy)phenyl]butan-1-ol,
2-amino-2-methyl-4-[4-(3-biphenyl-4-ylpropoxy)phenyl]butan-1-ol,
2-amino-2-methyl-4-[4-(3-biphenyl-4-ylpropionyl)phenyl]butan-1-ol,
2-amino-2-methyl-4-[3-methoxy-4-(4-phenylbutoxy)phenyl]butan-1-ol,
2-amino-2-methyl-4-[4-(5-phenylpentoxy)phenyl]butan-1-ol,
2-amino-2-methyl-4-[4-(5-phenylpentanoyl)phenyl]butan-1-ol,
2-amino-2-methyl-4-(4-hexyloxyphenyl)butan-1-ol,
2-amino-2-methyl-4-[4-(3-phenylpropoxy)phenyl]butan-1-ol,
2-amino-2-methyl-4-[4-(3-cyclohexylpropoxy)phenyl]butan-1-ol,
2-amino-2-methyl-4-[4-(5-cyclohexylpentanoyl)phenyl]butan-1-ol,
2-amino-2-methyl-4-[4-heptyloxyphenyl]butan-1-ol,
2-amino-2-[4-(3-benzyloxyphenoxy)-2-chlorophenyl]propan-1,3-diol,
2-amino-2-[4-(3-benzyloxyphenylthio)-2-chlorophenyl]propan-1,3-diol,
2-amino-2-methyl-5-[4-(3-benzyloxyphenoxy)-2-chlorophenyl]pentan-1-ol and
2-amino-2-methyl-5-[4-(3-benzyloxyphenylthio)-2-chlorophenyl]pentan-1-ol.

21. A pharmaceutical composition according to claim 1, in which the HMG-COA reductase inhibitor is atorvastatin and the amino alcohol compound having the formula (I) is
2-amino-2-[2-(4-octylphenyl)ethyl]propan-1,3-diol.

22. A pharmaceutical composition according to claim 1, wherein a ratio of said HMG-COA reductase inhibitor or a pharmacologically acceptable salt thereof or a pharmacologically acceptable ester thereof to said amino alcohol compound or a pharmacologically acceptable salt thereof or a pharmacologically acceptable ester thereof is 400:1 to 2000:1.

23. A pharmaceutical composition according to claim 1, wherein the pharmaceutical composition is for preventing or treating an autoimmune disease selected from the group consisting of systemic lupus erythematosus, rheumatoid arthritis, polymyositis, dermatomyositis, scleroderma, Behcet's syndrome, Crohn's disease, ulcerative colitis, inflammatory bowel disease, autoimmune hepatitis, a plastic anemia, idiopathic thrombocytopenic purpura, autoimmune hemolytic anemia, multiple sclerosis, autoimmune bullosis, psoriasis vulgaris, vasculitis syndrome, Wegener's granuloma, uveitis, idiopathic interstitial pneumonia, Goodpasture's syndrome, sarcoidosis, allergic granulomatous angitis, bronchial asthma, myocarditis, cardiomyopathy, aortitis syndrome, postmyocardial infarction syndrome, primary pulmonary hypertension, minimal change nephrotic syndrome, membranous nephropathy, membranoproliferative glomerulonephritis, focal glomerular sclerosis, crescentic glomerulonephritis, myasthenia gravis, inflammatory neuropathy, atopic dermatitis, chronic actinic dermatitis, acute polyarthritis, Sydenham's chorea, systemic sclerosis, adult-onset type diabetes mellitus, insulin dependent diabetes mellitus, juvenile diabetes mellitus, atherosclerosis, glomerular nephritis, tubulointerstitial nephritis, primary biliary cirrhosis, primary sclerosing cholangitis, fulminant hepatitis, viral hepatitis, GVHD, a rejection symptom caused by a transplantation of an organ, contact dermatitis and sepsis.

24. A pharmaceutical composition according to claim 1, in which
R¹ and R² are each a hydrogen atom,
R³ is a methyl group, an ethyl group or a hydroxymethyl group,
R⁴ is a hydrogen atom or a halogen atom,
R⁵ is a hydrogen atom, an unsubstituted phenyl group or a phenyl group substituted with from 1 to 3 substituents from Substituent group a,
Z is a single bond or a C₁-C₈ alkylene group, and n is an integer of 2.

25. A pharmaceutical composition according to claim 1, in which the HMG-COA reductase inhibitor is atorvastatin and the amino alcohol compound having the formula (I) is
2-amino-2-methyl-4-{1-methyl-5-[4-(4-methylphenyl)butanoyl]pyrrol-2-yl}butan-1-ol.

26. A pharmaceutical composition according to claim 1, in which the HMG-COA reductase inhibitor is atorvastatin and the amino alcohol compound having the formula (I) is
2-amino-2-methyl-4-{1-methyl-5-[4-(3,4-dimethylphenyl)butanoyl]pyrol-2 -yl}butan-1-ol.

27. A pharmaceutical composition according to claim 1, in which the HMG-COA reductase inhibitor is atorvastatin and the amino alcohol compound having the formula (I) is
2-amino-2-methyl-4-{1-methyl-5-[5-(4-methylphenyl)pentanoyl]pyrrol-2-yl}butan-1-ol.

28. A pharmaceutical composition according to claim 1, in which the HMG-COA reductase inhibitor is atorvastatin and the amino alcohol compound having the formula (I) is
2-amino-2-methyl-4-{1-methyl-5-[5-(3,4-dimethylphenyl)pentanoyl]pyrrol -2-yl}butan-1-ol.

29. A pharmaceutical composition according to claim 1, in which the HMG-COA reductase inhibitor is pravastatin and the amino alcohol compound having the formula (I) is
2-amino-2-methyl-4-{1-methyl-5-[4-(4-methylphenyl)butanoyl]-pyrrol-2-yl}butan-1-ol.

30. A pharmaceutical composition according to claim 1, in which the HMG-COA reductase inhibitor is pravastatin and the amino alcohol compound having the formula (I) is
2-amino-2-methyl-4-{1-methyl-5-[4-(3,4-dimethylphenyl)butanoyl]pyrrol-2-yl}butan-1-ol.

31. A pharmaceutical composition according to claim 1, in which the HMG-COA reductase inhibitor is pravastatin and the amino alcohol compound having the formula (I) is
2-amino-2-methyl-4-{1-methyl-5-[5-(4-methylphenyl)pentanoly]pyrrol-2-yl}butan-1-ol.

32. A pharmaceutical composition according to claim 1, in which the HMG-COA reductase inhibitor is pravastatin and the amino alcohol compound having the formula (I) is
2-amino-2-methyl-4-{1-methyl-5-[5-(3,4-dimethylphenyl)pentanoyl]pyrrol-2-yl}butan-1-ol.

33. A pharmaceutical composition according to claim 1, in which the HMG-COA reductase inhibitor is pravastatin and the amino alcohol compound having the formula (I) is
2-amino-2-[2-(4-octylphenyl)ethyl]propan-1,3-diol.

34. A kit comprising pharmaceutical preparation of a drug containing individually at least one HMG-COA reductase inhibitor and at least one amino alcohol compound having the following formula (I) or a pharmacologically acceptable salt thereof or a pharmacologically acceptable ester thereof: 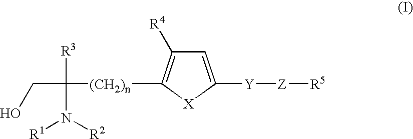
wherein
R¹ and R² are the same or different and each represents a hydrogen atom or a lower alkyl group,
R³ represents a lower alkyl group or a hydroxymethyl group,
R⁴ represents a hydrogen atom, a lower alkyl group, a lower alkoxy group or a halogen atom,
R⁵ represents a hydrogen atom, a halogen atom, a cyclohexyl group, an unsubstituted phenyl group or a phenyl group substituted with from 1 to 3 substituents from a Substituent group a,
X represents a vinylene group, an oxygen atom, a sulfur atom or a methylated nitrogen atom,
Y represents a single bond, an oxygen atom, a sulfur atom or a carbonyl group,
Z represents a single bond, an unsubstituted Cl-Ca alkylene group or a C₁-C₈ alkylene group substituted with from 2 to 8 fluorine atoms,
n represents an integer of 2 or 3 and
Substituent group is selected from the group consisting of a halogen atom, a cyano group, a lower alkyl group, a lower cycloalkyl group, a lower alkoxy group, a trifluoromethyl group, a phenyl group and a benzyloxy group.

35. A method for preventing or treating an autoimmune disease comprising administering to a mammal in need thereof a pharmaceutically effective amount of the pharmaceutical composition according to claim 1.

36. A method for preventing or treating an autoimmune disease comprising administering to a human in need thereof (i) at least one HMG-COA reductase inhibitor and (ii) at least one amino alcohol compound having the following formula (I), a pharmacologically acceptable salt thereof or a pharmacologically acceptable ester thereof: 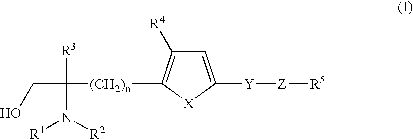
wherein
R¹ and R² are the same or different and each represents a hydrogen atom or a lower alkyl group,
R³ represents a lower alkyl group or a hydroxymethyl group,
R⁴ represents a hydrogen atom, a lower alkyl group, a lower alkoxy group or a halogen atom,
R⁵ represents a hydrogen atom, a halogen atom, a cyclohexyl group, an unsubstituted phenyl group or a phenyl group substituted with from 1 to 3 substituents from a Substituent group a,
X represents a vinylene group, an oxygen atom, a sulfur atom or a methylated nitrogen atom,
Y represents a single bond, an oxygen atom, a sulfur atom or a carbonyl group,
Z represents a single bond, an unsubstituted C₁-C₈ alkylene group or a C₁-C₈ alkylene group substituted with from 2 to 8 fluorine atoms,
n represents an integer of 2 or 3 and
Substituent group is selected from the group consisting of a halogen atom, a cyano group, a lower alkyl group, a lower cycloalkyl group, a lower alkoxy group, a trifluoromethyl group, a phenyl group and a benzyloxy group, wherein each of said HMG-COA reductase inhibitor and said amino alcohol compound are administered simultaneously, sequentially or separately on different occasions at a suitable time interval.

37. A method for preventing or treating an autoimmune disease comprising administering to a human in need thereof a pharmaceutically effective amount of the pharmaceutical composition according to any one of claims 1 to 33.
